SEQUENCE LISTING (1 sequence)

SEQ ID NO: 1 - protein, 6 aa, Human
EIHAGG

(19) United States
(12) Patent Application Publication
(10) Pub. No.: US 2003/0152576 A1
(43) Pub. Date: Aug. 14, 2003

(54) Interaction between cd14 and hbv components

(76) Inventors: Geert Leroux-Roels, Gent (BE); Peter Van Landschoot, Gent (BE)

Correspondence Address: Patricia A Kammerer, Howrey Simon Arnold & White, 750 Bering Drive, Houston, TX, 77057-2198, US

(21) Appl. No.: 10/168,032

(22) PCT Filed: Dec. 26, 2000
(86) PCT No.: PCT/EP00/13269

(30) Foreign Application Priority Data
Dec. 23, 1999 (EP) 99870283.1

(51) Int. Cl. A61K 39/42; C12Q 1/70

(52) U.S. Cl. 424/161.1; 435/5

(57) ABSTRACT

The present invention is based on the finding that HBV components (HBsAg) binds to CD14. LPS binding protein catalyses the attachment of HBsAg to CD14. The invention more particularly describes molecules having HBV receptor activity. The invention further describes molecules and compounds for preventing, treating or alleviating inflammatory reactions in patients. The invention also relates to new compounds directed against HBV infections, and methods for identifying them, including in vitro and in vivo model systems to do so. The invention further relates to new vaccine compositions directed against HBV. Additionally the invention also relates to the use of HBV components to treat inflammatory diseases.

### BACKGROUND TO THE INVENTION

The present invention relates to the field of Hepatitis B virus (HBV) infections. The invention describes molecules having receptor activity for HBV or HBV-derived components. The invention also relates to new compositions to treat HBV infection, as well as to new prophylactic and therapeutic vaccine compositions directed against HBV. Additionally the invention also relates to the use of HBV components to treat non-HBV inflammatory diseases. Further the invention relates to new compositions and methods for preventing, treating or alleviating inflammatory reactions.

Worldwide the Hepatitis B virus (HBV) causes more then 1 million deaths per year and about 350 million people are persistently infected with the virus. Most adult individuals will clear a primary infection, which can be asymptotic or can result in acute liver injury. However, approximately 5% will not resolve the primary infection and go on to a persistent infection. Vaccination is an effective way to prevent infection, but the giant virus reservoir in persistent carriers is a major obstacle to rapid eradication of the virus. The mechanism by which HBV establishes this persistent state is at present still unclear, but a failing immune response is clearly involved. In patients suffering from an acute resolving HBV infection strong and multispecific CTL responses towards envelope proteins are observed, whereas T helper cell and B cell responses against the envelope is less pronounced. In these patients there is a strong T helper cell response towards the capsidprotein (HBcAg) as well as a B cell response (Milich, 1997). In chronic HBV patients no or very weak pauci-specific responses towards envelope and core proteins are measured. A humoral response towards HbcAg is always discernible (Milich, 1997). In vitro studies have demonstrated a reduced capacity of PBMC from chronic infected persons to produce cytokines (Muller & Zielinski, 1990; Muller & Zielinski, 1992; Nagaraju et al., 1998; Vingerhoets et al., 1998). Administration of cytokines or cytokine induced by another (non-HBV) viral infection has a strong antiviral effect in mouse models of HBV infection (Cavanaugh et al. 1998)

The mechanism by which HBV enters cells has also not been elucidated. To infect a cell HBV must first bind to a host cell receptor via one or a combination of the three related viral membrane proteins L, M and S, which share 226 carboxy terminal amino acids. The M protein contains an additional pre-S2 (55 amino acids) sequence. The L protein contains S, pre-S2 and a pre-S1 sequence (108-119 amino acids). These membrane proteins are also found in the non-infectious surface antigen particles (HBsAg), which are present in circulation. Like virions such particles contain predominantly the S protein, but smaller amounts of M and hardly any L protein (Gerlich and Bruss, 1993). The reason for the existence of or the possible advantage the production of HBsAg may represent for the virus remains elusive until today. Recombinant expression of only the S protein results in assembly and secretion of non-infectious surface antigen particles. Such viral like particles (VLP's) have been used widely to identify cellular receptors. Initially the preS2 region was considered to be involved in the attachment process. Binding to polymerized human serum albumin, an unusual glycan structure, fibronectin and the transferrin receptor were reported. In recent years it has been suggested that the preS1 domain contains the attachment site. Binding to glyceraldehyde-3-phosphate-dehydrogenase, an IgA receptor, interleukin 6 and asialoglycoprotein were demonstrated. Attachment of the S protein to annexin V and apolipoptotein H has also been observed. (Imai et al., 1979; Franco et al., 1992; Gerlich et al., 1993; Budkowska et al., 1995; Petit et al., 1992; Pontisso et al., 1992; Neurath et al., 1992; Treichel et al., 1994; Hertogs et al., 1993; Mehdi et al., 1994).

Hepatocytes are believed to be the major natural host cells for Hepatitis B virus (HBV), but viral DNA and viral proteins have been detected in many other tissues and cell types. (Blum et al., 1983; Dejean et al., 1984; Zeldis et al., 1986; Harrison et al., 1990; Neurath et al., 1990; Mason et al., 1993). It has been postulated that cells of the lymphoid system may initially support replication (Korba et al., 1989). Keeping in mind the principal routes of HBV transmission (via blood contacts and mucosal surfaces), it seems not unlikely that like for HIV, the first cells infected by HBV are indeed monocytes/macrophages or T and B cells. Infection implies the presence of HBV specific receptors on the membrane of these cells.

It is the aim of the present invention to identify molecules interacting with HBV or HBsAg particles ad their uses.

The aim of the present invention is to provide molecules having HBV receptor activity and their use.

Another aim of the invention is to provide compositions for preventing, treating or alleviating HBV infections.

Another aim of the invention is to provide new vaccine compositions and methods for preventing, treating or alleviating HBV infections.

Another aim of the invention is to provide compositions and methods for preventing treating or alleviating inflammatory reactions.

Another aim of the invention is to provide screening processes for identifying compounds interfering with HBV components.

It is another aim of the invention to provide methods for preparing compositions including the compounds of the invention.

It is a further aim of the invention to provide cellular hosts to be used as a model system for testing potential drugs against HBV infection and to provide for assays which involve the use of this host.

Another aim of the present invention is to provide mammalian non-human transgenic animals, and for the use of these transgenic animals as a model system for testing potential drugs.

All the aims of the present invention have been met by the embodiments as set out below.

### DETAILED DESCRIPTION OF THE INVENTION

To identify HBV or HBsAg receptors on PBMC, the possible interaction of HBsAg with PBMC was investigated by fluorescence-activated cell sorting (FACS), using biotinylated recombinant HBsAg particles, which only contain S protein (b-rHBsAg). It is reported here that such particles bind almost solely to monocytes and not to T-cells, while some recognition of B-cells is observed. However, nothing is known yet on by which molecules (cellular receptor or others) HBsAg particles bind to the cell.

Data presented in the present invention clearly demonstrate that HBsAg particles, which contain only the S protein, specifically bind to monocytes through interaction with CD14 molecules. 1) Attachment of HBsAg is restricted to the CD14 positive cells of PBMC. 2)THP-1 and CHO cells which do not express CD14 also do not bind HBsAg efficiently. Highly enhanced attachment of HBsAg to these cells can be obtained after induction of CD14 by 1,25-VitD3 treatment or by transfection of CD14 cDNA, respectively. 3) Soluble CD14 can reduce the binding of HBsAg to the cell membrane of CD14 positive cells. 4) Binding of HBsAg is efficiently blocked by several CD14-specifc mAbs. 5) HBsAg can reduce the binding of CD14-specific mAbs to the cells.

Additionally, the present inventors surprisingly found that attachment of HBsAg to CD14 is strongly enhanced by the LPS-binding protein (LBP).

The present inventors also were able to show that HBsAg inhibits LPS and II-2 induced activation of monocytes as demonstrated by the reduced secretion of pro-inflammatory cytokines IL-1α, TNFβ or IL-8.

Expression of CD14 by human hepatocytes has recently been demonstrated (Hetherington et al., 1999; Su et al., 1999). This raises the possibility that HBV through its S protein can attach to hepatocytes using the same receptor. Circulating HBsAg might, through the interaction with LBP and CD14 prevent or even suppress the activity of monocytes and macrophages which adds to the development of chronic hepatitis B. Furthermore, due to the anti-inflammatory potential of HBsAg, this particle can be used to treat non-HBV inflammatory diseases.

Consequently, in a first embodiment, the present invention relates to a method for determining the binding between CD14 and Hepatitis B virus components comprising the use of:

a) CD14, or a mutein, or a fragment of CD14 which is able to bind to HBV antigens or particles,

b) HBV components,

c) a ligand or antisense peptide of the molecules mentioned in a) or b),

d) an antibody raised against any of the molecules mentioned in a) or b),

e) an antibody raised against the ligand mentioned in c),

f) an anti-idiotype antibody raised against the antibody mentioned in d), or,

g) an anti-idiotype antibody raised against the antibody menitioned in e).

Human CD14 (hCD14) is predominantly expressed on cells of the myelomonocytic lineage including monocytes, macrophages and Langerhans cells (Goyert et al., 1986; Ferrero et al., 1990). hCD14 is also expressed at lower levels on neutrophils and B cells (Labeta et al., 1991). Recently CD14 expression by human hepatocytes has been demonstrated (Hetherington et al., 1999; Su et al., 1999). The mature protein contains 356 amino acids; a signal sequence of 19 aa is removed during processing. The molecular weight of the polypeptide is 35773, while on SDS-PAGE it is 53-55 kDa. CD14 is anchored to the cell membrane by linkage to glycosylphosphatidylinositol (GPI-anchor; Haziot et al., 1988) and contains 4 N-linked carbohydrates. O-linked glycosylations are present as well (Stelter et al., 1996). At least two soluble forms of CD14 (sCD14) have been described. One form is produced by shedding of CD14 expressed on the cell membrane. A second form is released from cells before addition of the GPI anchor (Butler et al., 1995). Several mAbs, like My4 and M(P9), directed against CD14 and having different characteristics have been isolated and described. Many of these are commercially available. The recognition of CD14 by such mAbs may differ from cell type to cell type, although CD14 mRNA from different cell types show identical nucleotide sequences. Mouse (mCD14) has been sequenced and shows less then 70% homology with hCD14 (Setoguchi et al., 1989;Matsuura et al., 1989; Ferrero et al., 1990). Tissue distribution of mCD14 is different when compared to hCD14 (Fearns et al., 1995).₁₃ mCD14 for example is expressed on peritoneal monocytes but not on blood monocytes.

In the present invention, the term “CD14” relates to the full-length polypeptide and may be from human or any non-human mammalian origin.

The term “mutein derived from CD1 4” which is able to bind to HBV components refers to a derivative or homologue of naturally occurring CD14 which may be experimentally identified to bind as well as CD14 to HBV components under the following conditions. Wells of an ELISA plate are coated with the purified mutein of CD14 (1 μg/ml in PBS). After blocking with 0.1% BSA in PBS, biotinylated HBsAg (1 μg/ml in PBS-0.1% BSA) is added to the wells. After incubation for 1.5 h the wells are washed and are incubated with alkaline phophatase-conjugated streptavidine. After washing, substrate solution (1 mg/ml Na p-nitrophenyl in diethylamine buffer pH 9.8) is added. The absorbance is measured at 405 nm at appropriate times.

The term “fragment of CD14” which is able to bind to HBV components refers to a shorter version of CD14 or a mutein thereof and may be experimentally indentified to bind as well as CD14 to HBV components under the following conditions. Wells of an ELISA plate are coated with the purified shorter version of CD14 (1 μg/ml in PBS). After blocking with 0.1% BSA in PBS, biotinylated HBsAg (1 μg/ml in PBS-0.1% BSA) is added to the wells. After incubation for 1.5 h the wells are washed and are incubated with alkaline phophatase-conjugated streptavidine. After washing, substrate solution (1 mg/ml Na p-nitrophenyl in diethylamine buffer pH 9.8) is added. The absorbance is measured at 405 nm at appropriate times.

In the present invention, the term Hepatitis B virus (HBV) “components” refers to the HBV virus, HBV molecules, particles, antigens, peptides or more specific fragments of the Hepatitis B viral or envelope polypeptides.

Preferred “HBV components” of the invention are HBV particles, preferably HBsAg particles.

The term “HBV particles” refers to non-infectious surface antigen particles (HBsAg) containing the viral membrane proteins and are found in the circulation. HBsAg is a macromolecular particle that is composed of protein, carbohydrates (in the form of glycoproteins) and lipids. The lipid moiety is of host origin and represents approximately 25% of the particle mass. Like virions such particles contain predominantly the S protein, but smaller amounts of M and hardly any L protein. Frequently used HBV particles are the recombinant non-infectious surface antigen particles (rHBsAg) produced in S. cerevisiae and which are commercially available by Smithkline Beecham Biologicals (Rixensart, Belgium) and Merck Sharpe and Dohme (MSD, USA). rHBsAg particles contain only the S protein.

According to the invention, HBV particles, can be composed of the naturally occuring mixture of lipids (phosphatidyl-choline, cholesterol, cholesterolesters) and proteins (S, M, L proteins). However the present invention also relates to derivates of HBV particles wherein the lipid moiety of said particle is replaced by other lipids or mixtures of lipids such as phosphatidyl-glycerol, phosphatidyl-serine and phosphatidyl-inositol. Other possible derivatives of HBV particles have a protein moiety, which is modified, for instance for better binding to CD14. This can be achieved for instance by changing amino acid residues or region which may be important for binding to CD14.

It is to be understood that in the context of the present invention the term “ligand” can mean “receptor ligand” or can refer to any ligand, which is able to bind to CD14 in a specific way.

The present inventors surprisingly found that a serum protein, named LPS Binding Protein (LBP) catalyses the attachment of HBsAg to CD14. According to the invention, LBP is a preferred ligand of HBV components. The present inventors demonstrated that LBP binds to HBsAg after which this complex binds to CD14. Subsequently, LBP detaches from the HBsAg-CD14-LBP complex. The identification of said newly found interaction(s) is also part of the invention.

According to a preferred embodiment the invention also relates to a method for determining the binding between HBV components, preferably HBsAg, and LPS binding protein comprising the use of:

a) HBV components,

b) an antibody raised against any of the moleculs mentioned in a), or,

c) an anti-idiotypic antibody raised against the antibody mentioned in b).

Lipopolysaccharide (LPS)-binding protein (LBP) is a 60-kDa glycoprotein found in plasma of all species studied so far. In humans it is normally present at 3-7 μg/ml. It is an acute phase protein synthesized principally in hepatocytes with smaller amounts in other tissues. LBP is the prototypical member of a four-protein family of structurally similar proteins that includes LBP, bactericidal/permeability-increasing protein (BPI), cholesterol ester transfer protein (CETP) and phospholipid transfer protein (PLTP). LBP binds to the lipid A moiety of LPS, but is also able to bind phopsholipids like phophatidyl-serine (PS), phosphatidyl-choline (PC), phosphatidyl-ethanolamine (PE) and phosphatidyl-inositol (PI). LBP is best known for its ability to catalyze the transfer of LPS to the major LPS receptor CD14 and to phospholipid membranes. The principal mechanism appears to be the ability to dissociate LPS aggregates into LPS monomers bound to LBP and to deliver these to CD14. LBP has also been shown to transfer PS and PI to CD14 (Ulevitch and Tobias, 1999).

According to a preferred embodiment the invention thus also relates to the use of LBP or a derivative or a fragment thereof, as a preferred ligand of HBV components (e.g. HBsAg particles), in a method for determining the binding between CD14 and Hepatitis B components.

The property of binding to CD14 can be tested using the BIAcore system in which the binding of the ligand to adsorbed CD14 is monitored (Malmqvist, 1999).

It is to be noted that binding conditions given in this application are given as an example, and can be modified by the person skilled in he art within limits such that the function of binding remains equivalent to the one defined under the above-mentioned conditions.

Preferred illustrations of other ligands include for instance antisense peptides as defined below, as well as possible drugs, which specifically bind to CD14 present on the cell's plasma membranes.

The term “antisense peptide” is reviewed by Blalock (1990) and by Roubos (1990). In this respect, the molecular recognition theory (Blalock, 1990) states that not only the complementary nucleic acid sequences interact but that, in addition, interacting sites in proteins are composed of complementary amino acid sequences (sense-receptor ligand or sense-antisense peptides). Thus, two peptides derived from complementary nucleic acid sequences in the same reading frame will show a total interchange of their hydrophobic and hydrophilic amino acids when the amino terminus of one is aligned with the carboxy terminus of the other. This inverted hydropathic pattern might allow two such peptides to assume complementary conformations responsible for specific interaction.

The expression “anti-idiotype antibodies raised against the antibody mentioned in d)”by Gheuens and MacFarlin (1982). Monoclonal anti-idiotypic antibodies have the property of forming an immunological complex with the idiotype of the monoclonal antibody against which they were raised. In this respect the monoclonal antibody is referred to as Ab1, and the anti-idiotypic antibody is referred to as Ab2. Ab2 to CD14, or to muteins or fragments derived thereof can thus recognise the receptor binding site on HBV, and can consequently block this HBV binding site, thereby preventing HBV attachment and infection.

On the other hand, the expression “anti-idiotype antibodies raised against the antibody mentioned in e)” refers to monoclonal antibodies raised against the antigenic determinants of the variable region of the monoclonal antibodies raised against a ligand or antisense peptide of the molecules described in a) or b). In this respect the monoclonal antibody is referred to as Ab3, and the anti-idiotypic antibody is referred to as Ab4. Ab4 to a ligand or antisense peptide of the molecules of a) can thus recognise the ligand or antisense peptide binding site on CD14 and can consequently block this binding site, thereby preventing or interfering with HBV attachment and infection.

The monoclonal antibodies according to the invention are preferably human monoclonal antibodies or humanised versions of monoclonal antibodies.

The expression “determining the binding” in claim 1 refers to all methods known in the art to possibly assess the binding between CD14 and HBV components. For instance “determining the binding” may be experimentally defined under the following. Wells of an ELISA plate are coated with purified CD14 (1 μg/ml in PBS). After blocking with 0.1% BSA in PBS, biotinylated HBsAg (1 μg/ml in PBS) is added to the wells. After incubation for 1.5 h the wells are washed and are incubated with alkaline phophatase-conjugated streptavidine. After washing, substrate solution (1 mg/ml Na p-nitrophenyl in diethylamine buffer) is added. The absorbance is measured at 405 nm at appropriate.

The expression “the use of” refers to any use in any method known to the one skilled in the art. This can be for instance the use in binding assays, ELISA's, FACS analysis, Western Blots, Line immuno-assay (Lia), BIAcore real time detection system.

The invention also relates to a composition for preventing, treating or alleviating HBV infections comprising as an active substance at least one of the following:

a) CD14 or a mutein or fragment thereof which binds to HBV components,

b) a ligand or antisense peptide of the molecules mentioned in a),

c) an antibody raised against the molecules mentioned in a),

d) an antibody raised against the ligand or antisense peptide mentioned in b),

e) an anti-idiotype antibody raised against the antibody mentioned in c),

f) an anti-idiotype antibody raised against the antibody menioned in d),

g) fragments of HBV envelope proteins which bind to CD14, and,

h) antibodies specifically recognizing the fragments mentioned in g).

According to the invention, the peptides or polypeptides referred to by a), the antibodies directed to HBV components referred to by h), the antibodies referred to by d) and the anti-idiotypic antibodies referred to by e) herein, compete with CD14 for the CD14-binding site on HBV or on HBV components. These polypeptides, muteins and fragments, monoclonal or polyclonal antibodies and anti-idiotypic antibodies are (pharmacologically) active insofar as they prevent virus attachment and infection or prevent HBV components from attaching to the cells.

According to the invention, the ligand or antisense peptides referred to by b), the antibodies referred to by c) and the anti-idiotype antibodies referred to by f) herein have the properties of competing with the virus or with the HBV components for the HBV binding site present on CD14. These ligand or antisense peptides, monoclonal or polyclonal antibodies and anti-idiotype antibodies are (pharmacologically) active insofar as they block the Hepatitis B virus or the HBV component binding site on the CD14 molecule(s) present on the cell membrane and thus protect the cell from virus attachment and infection or HBV component attachment.

According to the invention the anti-idiotype antibodies referred to by e) herein, recognise the binding site on HBV or on HBV components and can consequently block this binding site of the virus or the HBV component, thereby preventing virus attachment and infection or HBV component attachment.

Analogous, anti-idiotype antibodies referred to by f) herein, are competitors with the virus or the HBV components and are (pharmacologically) active insofar as they block the HBV or HBV components binding site of CD14 on the cell and protect the cell from attachment of the virus or HBV components.

The expression “the HBV binding site” more preferably relates to the HBsAg or/and rHBsAg binding site.

The expression “fragments of HBV envelope proteins” relates to shorter versions of the HBV envelope proteins insofar as they contain the CD14 binding site. In a preferred embodiment, they contain the antigenic determinants recognised by antibodies. These antibodies may specifically interfere with the binding between CD14 and HBV and thus prevent virus attachment and infection or prevent HBV components from attaching to the cells.

Patients, who suffer from a HBV infection have been shown to contain HBsAg particles in the circulation. These particles could be responsible for the suppression of the immune system by binding to CD14, which is expressed on antigen-presenting cells. Therefore, administration of (soluble) CD14 or fragments thereof comprising the HBsAg binding fragment could prevent binding of HBsAg particles to the cells.

The present inventors found that HBsAg particles bind to a region of CD14 comprising the sequence E-I-H-A-G-G (SEQ ID NO 1) which corresponds to amino acids 39 to 44 of the human CD14 protein. Howeverit is not excluded that also other fragments in CD14 can be involved in binding.

According to a preferred embodiment the present invention thus relates to a composition for preventing, treating or alleviating HBV infections comprising as an active substance a CD14 fragment which binds to HBV components, preferably HBsAg, wherein said fragment is a peptide consisting of 6 to 50 amino acids, preferably 10, 12, 15, 20, 25, 30, 35, 40, 45 or 45 amino acids, comprising the sequence as represented in SEQ ID NO 1. Experiments with mutant human CD14 molecule(s) showed that important residues in this sequence are at positions 39, 40, 41,43 and 44 of CD14.

Also according to the invention are compositions wherein the fragments of CD14 are derived from non-human CD14 molecules, wherein said fragments comprise the HBsAg binding region. It is expected that said HBsAg binding region is located in a region corresponding to the HBsAg binding region in human CD14. However, because said CD14 molecules are derived from other organisms, it is possible that the amino acid sequence will be different from the sequence as represented in SEQ ID NO 1.

However since the present invention relates to the general concept that HBV components, e.g. HBsAg bind to CD14 on antigen presenting cells, the invention also relates to the fragments of CD14 which bind to HBsAg but isolated from non-human organisms.

The invention further relates to isolated polypeptides consisting of 6 to 50 amino acids comprising the sequence as represented in SEQ ID NO 1. Said polypeptides can be used for the preparation of a medicament for preventing, treating or alleviating HBV infections or inflammatory reactions.

In a further preferred embodiment, the present invention relates to a composition for preventing, treating or alleviating HBV infections comprising as an active substance an antibody against a ligand of HBV components, preferably HBsAg particles, wherein said ligand is the LPS binding protein.

The LPS binding protein catalyses the binding of HbsAg to CD14. By complexing the LPS binding protein with an antibody aginst this ligand of HBsAg, attachment of HbsAg can be inhibited. Several mAbs directed against the LPS binding protein and having different characteristics have been isolated and described. Many of these are commercially available.

According to a further preferred embodiment, the present invention relates to a composition for preventing, treating or alleviating HBV infections comprising as an active substance a peptide which blocks the binding of the LPS binding protein to HBV components.

Examples of peptides which block the binding between LPS and LBP are for instance described in Scott et al. (2000) and can be used in the present invention.

Since the LPS binding protein catalyses the binding of HbsAg to CD14, complexing of the LPS binding protein with such peptides, can decrease the binding of HbsAg to CD14.

The invention further relates to vaccine compositions for preventing, treating or alleviating HBV infections comprising as an active substance at least one of the following:

a) ligands or antisense peptides as defined in claim 1 or antibodies directed against these ligands or antisense peptides,

b) anti-idiotype antibodies raised against the receptor ligands or antisense peptides as defined in claim 1,

c) muteins or fragments of CD14 as defined in claim 1 or antibodies directed against these muteins or fragments,

d) immunogenic fragments of HBV antigens which interact with CD14 fragments comprising epitopes of antigens of HBV which bind to CD14, or,

e) antibodies specifically recognizing the fragments or d).

The term “immunogenic fragments of HBV antigens” refers to fragments, which are able to raise antibodies against the HBV antigens.

In a more preferred embodiment the invention relates to a vaccine composition for preventing, treating or alleviating HBV infections comprising as an active substance at least one of the following:

a) an antibody raised against a ligand of HBV components as defined in claim 1, wherein said ligand is the LPS binding protein, or

b) an antibody raised against CD14.

Passive immunisation of Hepatitis B infected patients with antibodies directed against either CD14 or the LPS binding protein might prevent binding of HBV components (preferably HBsAg) to CD14 positive antigen presenting cells. By preventing this attachment, the activity of these cells will no longer be suppressed. These cells may now help to establish a potent anti-HBV immune response to clear the HBV infection.

The composition according to the invention can be a pharmaceutical, diagnostic or vaccine composition.

CD14 has been shown to be involved in many immune processes. First of all, CD14 is the major LPS receptor (Wright et al., 1990), but binds also to other microbial products (Newman et al., 1995; Soell et al., 1995; Heumann et al., 1994; Kusunoki et al., 1995).

These interactions provoke the release of cytokines (TNFα, IL-1 and IL-6), which trigger acute phase responses (Moshage, 1997). However, engagement of CD14 does not necessarily result in production of these cytokines. It has recently been shown that monocytes phagocytose apoptotic T-cells through interaction with CD14 without release of inflammatory cytokines (Devitt et al., 1998). Recent work also suggest that this interaction generates signals that actively suppress pro-inflammatory signals (Voll, 1997; Fadok et al., 1998). CD14 is also supposed to be involved in the regulation of monocyte apoptosis, because increased or decreased expression precedes survival or apoptosis, respectively (Heidenreich et al., 1997). Monocyte and T- and B cell activation is probably also dependent on interactions of CD14 with unknown receptors. Engagement of CD14 by specific mAbs on monocytes results in suppression of LPS and IL-2 induced₁₃ monocyte responses (Bosco et al., 1997), terminates T-cell proliferation and inhibits synthesis of IgG's (Lue et al., 1991; Jabara et al., 1994).

Vaccine compositions of the prior art are for instance, described in Ellis, R. W, 1993 and further in Blumberg, 1997; Papaevangelou, 1998; Adkins and Wagstaff, 1998.

The vaccine compositions according to the present invention comprise new active substances which have never been used in HBV treatment.

Vaccine compositions may be used either prophylactic or therapeutic.

The present inventors were able to show that HBsAg inhibits LPS induced activation of monocytes as demonstrated by the reduced secretion of pro-inflammatory cytokines IL-1β and TNFα. This anti-inflammatory potential could be involved in the establishment of a persistent virus infection.

These observations raise the possibility that

a) preventing the attachment of HBsAg to CD14 in infected patients will result in the release of inflammatory cytokines. These cytokines could help to clear the infection,

b) a HBV derived vaccine which does not bind to CD14 is more likely to be more immunogenic,

c) because of the anti-inflammatory potential of HbsAg, these particles can be used as a therapeutic agent for treating inflammatory reactions and endotoxine-induced responses (shock).

The invention thus relates to a composition for treating, preventing or alleviating inflammatory reactions in patients comprising the use of HBV components, preferably HBsAg. Optionally said HBV components are administered in combination with LPS binding protein.

According to the invention, said inflammatory reactions are preferably not related with HBV infections and are therefore also named non-HBV inflammatory reactions.

The binding of HBsAg to CD14 positive cells (monocytes, macrophages) results in a reduction in the release of pro-inflammatory molecules. As the binding of HBsAg to CD14 is strongly enhanced by the LPS binding protein, co-administration of this ligand could increase the efficacy of the therapy.

The invention further relates to the use of HBV components, preferably HBsAg particles, (optionally in combination with LPS binding protein) for treating, preventing or alleviating inflammatory reactions

In the alternative, the invention relates to the use of HBV components, preferably HBsAg particles (optionally in combination with LPS binding protein) for the manufacture of a medicament for treating, preventing or alleviating inflammatory reactions.

The polypeptides of the present invention may be produced by recombinant expression in prokaryotic and eukaryotic host cells such as bacteria, yeast, fungi or mammalian cells, which have been previously transformed by a recombinant vector coding for the polypeptides of the invention. It is expected that those of skill in the art are knowledgeable in the numerous expression systems available for expression in these systems.

A “recombinant” vector will comprise a recombinant DNA molecule comprising a “heterologous sequence” meaning that said recombinant DNA molecule will comprise a sequence originating from a foreign species, or, if from the same species, may be substantially modified from its original form. For example, a promoter operably linked to a structural gene which is from a species different from which the structural gene was derived, or, if from the same species, may be substantially modified from its original form. It should be understood that the “heterologous sequence” can be the complete CD14 gene or nucleic acid sequences encoding muteins or fragments or peptides derived from CD14 and may be from human or any non-human mammal origin. The “heterologous sequence” can also be a nucleic acid sequence coding for HBV components, or fragments thereof or peptides derived thereof.

The polypeptides and peptides of the invention can also be prepared by classical chemical synthesis.

The synthesis can be carried out in homogeneous solution or in solid phase. For instance, the synthesis technique in homogeneous solution which can be used is the one described by Houbenweyl in the book entitled “Methode der organischen chemie” (Method of organic chemistry) edited by E. Wunsh, vol. 15-I et II, THIEME, Stuttgart 1974.

The polypeptides of the invention can also be prepared in solid phase according to the methods described by Atherton and Shepard in their book entitled “Solid phase peptide synthesis” (IRL Press, Oxford, 1989).

All these methods are well known to those skilled in the art.

The antisense peptides can be prepared as described in Ghiso et al. (1990). By means of this technology it is possible to logically construct a peptide having a physiological relevant interaction with a known peptide by simple nucleotide sequence analysis for complementarity, and synthesize the peptide complementary to the binding site.

The monoclonal antibodies of the invention can be produced by any hybridoma able to be formed according to classical methods from splenic lymphocytes of an animal, particularly of a mouse or rat, immunized against the CD14 or muteins thereof or ligand or antisense peptides defined above, on the one hand, and of cells of a myeloma cell line on the other hand, and to be selected by the ability of the hybridoma to produce the monoclonal antibodies recognizing the polypeptides which has been initially used for the immunization of the animals.

The antibodies involved in the invention can be labeled by an appropriate label of the enzymatic, fluorescent or radioactive type or any label known in the art.

The monoclonal antibodies according to this embodiment of the invention may be humanized versions of mouse monoclonal antibodies made by means of recombinant DNA technology, departing from parts of mouse and/or human genomic DNA sequences coding for H and L chains or from cDNA coding for H and L chains.

Alternatively the monoclonal antibodies according to this embodiment of the invention may be human monoclonal antibodies. These antibodies according to this embodiment of the invention can also be derived from human peripheral blood lymphocytes (PBL) of patients infected with HBV, or vaccinated against HBV. Such human monoclonal antibodies are prepared, for instance, by means of human peripheral blood lymphocytes repolution of severe combined immune deficiency (SCID) mice (for review, see Duchosal et al. 1992) The anti-idiotype antibodies raised against CD14, or against its muteins, or against the fragments as defined above, or anti-anti-idiotype antibodies raised against CD14 as defined above can be prepared as mentioned above.

The anti-idiotype antibodies raised against ligand or antisense peptides as as defined above, or anti-anti-idiotype antibodies raised against ligand or antisense peptides can be prepared as mentioned above.

In this respect, according to another embodiment, the present invention relates to methods and processes for screening new anti-hepatitis drugs.

Life threatening inflammatory reactions can be induced by trauma and bacterial, viral and fungal infections. For example sepsis is such an important medical life-threatening syndrome characterized by chills, profuse sweating, fever, weakness or hypotension, leukopenia, intravascular coagulation, multiple organ failure and often death (Ulevitch et al., 1990). Lipopolysaccharides (LPS) from gram-negative bacteria are well known inducers of sepsis, but cell wall substances from gram-positive and fungal origin can cause this syndrome as well. Cytokines like TNF play an important role in the development of the disease (Beutler et al., 1985). Other LPS-like induced diseases include ARDS, acute pancreatitis, acute and chronic liver failure, intestinal and liver transplantation, inflammatory bowel disease, graft vs. host disease following bone marrow transplantation and tuberculosis.

According to another embodiment, the invention relates to a host cell transformed with a nucleic acid sequence coding for muteins of CD14 or parts of CD14 which bind to HBV components, with said host expressing said insert polypeptides or peptides on its surface. Preferred parts or fragments of CD14 comprise the sequence as represented in SEQ ID NO 1 and as described earlier.

Host cells expressing CD14 have been described for instance by Golenbock et al. (1993), Jack et al. (1995), Kravchenko et al. (1996) and Devitt et al. (1998).

It should be understood that the host cell has been transformed with a recombinant vector as defined earlier.

The invention also relates to methods for screening drugs which can be used in the field of HBV infection and in the field of inflammatory reactions in patients as defined above.

More specifically, the invention relates to a method for identifying compounds interfering with the interaction between CD14 and HBV components comprising the steps of:

a) contacting the compound to be screened with a complementary set of molecules as defined in claim 1, and,

b) detecting the binding affinity of said complementary set of molecules as defined in claim 1 in the presence or absence of said compound to be tested.

The invention also relates to a method for identifying compounds interfering with the interaction between CD14 and HBV components comprising the steps of:

a) contacting the compound to be screened with a cellular host as described above, and,

b) detecting the formation of a complex between said compound and said CD14 molecule or a mutein or fragment thereof,

c) providing HBV components optionally in combination with LBP, and,

d) detecting the interference of said compound with the binding of HBV components with said CD14 molecule or a mutein or a fragment thereof.

The invention also relates to a method for identifying compounds interfering with the interaction between HBV components and LPS binding protein comprising the steps of:

a) contacting a compound to be screened with a host as defined above, HBV components and LPS binding protein,

b) detecting the interference of said compound with the binding of HBV components with said CD14 molecule or a mutein or a fragment thereof, and,

c) detecting a complex formation between said compound and HBV components or LPS binding protein

The invention further relates to the above-mentioned method, wherein said detection steps in b), c) or d) is a quantitative detection step to determine the affinity of binding said compound.

The invention also relates to the compounds obtainable by any of the above described methods. These compounds may be further modified for use in a delivery system. Said modifications may include the binding of the compounds to beads or to a targeting molecule such as an antibody, eventually by means of a spacer molecule. Also any other method for modification of molecules known in the art for purposes of delivery can be applied.

The term “delivery of a compound” relates to methods of delivery or administration of such compound via oral, enteral or parenteral route such as for instance tablets, capsules, ampoules for injection or any other method known for purpose of delivery. The compound may as well be delivered or administered by means of controlled-release devices and methods.

The invention also relates to the use of a compound as described above for preventing, treating or alleviating inflammatory reactions, or in the alternative for the preparation of a medicament for preventing, treating or alleviating inflammatory reactions in patients.

The invention further relates to the use of a compound as described above or a molecule as defined in claim 1 or 2 for preventing, treating or alleviating HBV infections, or in the alternative for the preparation of a medicament for preventing, treating or alleviating HBV infections.

According to yet another embodiment, the invention relates to a host cell for use as a model system for testing potential drugs which are able to exert at least one of the following functions:

a) modulate the binding or the affinity of binding of HBV components to said host cell,

b) modulate the expression of CD14 or its muteins or its fragments on the surface of said host cell,

c) modulate the activity of HBV components or the activity of CD14 or its muteins or fragments thereof, or

d) interfere with the replication of HBV in said host cell.

A preferred example of said model system is an in vitro assay to test possible drugs which can be used in the field of HBV infection or inflammation reactions.

The invention also relates to a mammalian non-human transgenic animal able of being infected by HBV and possibly suffering from hepatocellular damage caused by the consecutive or inducible expression of a nucleic acid encoding a CD14 mutein or a part of CD14 introduced into the genome of said transgenic animal, as a model system for testing potential drugs which are able to exert at least one of the following functions:

a) modulate the binding or the affinity of binding of HBV to a cell of said transgenic animal,

b) modulate the expression of CD14 or its muteins or its fragments on the surface of a cell of said transgenic animal,

c) modulate the activity of HBV components or the activity of CD14 or its muteins or fragments thereof, or

d) interfere with the replication of HBV in said transgenic animal.

According to a further embodiment, the invention relates to a mammalian non-human transgenic animal, said animal expressing HBV, as a model system for testing potential drugs which are able to exert at least one of the following functions:

a) modulate the binding or the affinity of binding of HBV components to CD14 expressing cells of said transgenic animal,

b) modulate the expression of CD14 on the surface of a cell of said transgenic animal, or,

c) moldulate the activity of HBV components or the activity of CD14

A transgenic non-human mamalian animal can be prepared according to the protocol described in International Review of Cytology, Gordon J W, vol. 115, p. 171-222 (1989). Transgenic mice expressing CD14 have been described by Ferrero et al. (1993), Hetherington et al. (1998) and Tamura et al. (1999).

The invention also relates to a method for identifying potential HBV drugs comprising the use of a mammalian non-human transgenic animal wherein the animal CD14 gene is replaced by a human CD14 gene or wherein at least part of a human CD14 gene is introduced. Also the componds identified by this method are part of the invention.

According to another embodiment, the invention relates to a method for the production of a composition comprising a compound identified by a method as defined above and possibly mixing said compound with a pharmaceutically acceptable carrier.

Pharmaceutical compositions according to the present invention, and for use in accordance to the present invention, may comprise, in addition to the compound, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration. Those of relevant skill in the art are well able to prepare suitable solutions.

The invention, now being generally described, will be more readily understood by reference to the following examples, which are included merely for purposes of illustration of certain aspects and embodiments of the present invention and are not intended to limit the invention. All of the references mentioned herein are incorporated by reference.

### BRIEF DESCRIPTION OF FIGURES

FIG. 1: Biotinylation of rHBsAg interferes with the binding of mAbs specific for the major antigenic region.

Elisa plates were coated with two-fold dilutions of rHBsAg(◯) and b-rHBsAg (●) After blocking with BSA, human mAbs F47B, F9H9, anti-a and the mouse mAb antid-d were allowed to bind to the particles. mAbs were detected with either goat-anti-mouse or goat-anti-human-peroxidase labeled antibodies.

FIG. 2: Attachment of b-rHBsAg to CD14 positive PBMC.

Cells were incubated for 1 h with different concentrations of b-rHBsAg in 200 μl 2% HS-HBSS, washed twice with the same buffer and incubated with anti CD14-FITC (clone P9), anti CD19-FITC or anti CD3-FITC and Strep-PE. After two washes cells were analyzed. Percentage of b-rHBsAg positive CD14 (black bars), CD19 (grey bars) or CD3 (white bars) positive cells was determined. A typical dot blot staining pattern for PBMC is shown on the right. Upper panel shows PBMC stained with SAPE only, the lower panel shows staining of PBMC incubated with b-rHBsAg followed by SAPE.

FIG. 3: Non-biotinylated rHBsAg competes with attachment of b-rHBsAg to monocytes. PBMC were incubated with different amounts of rHBsAg in 200 μl 2% HS-HBSS. After 1 h b-rHBsAg was added and the cells were incubated for another hour. After two washes cells were stained with Strep-PE, washed twice and analysed. Median fluorescence was determined. The data shown represent the average of three separate experiments. Error bars represent SD.

FIG. 4: Ca²⁺ and Mg²⁺ interfere with attachment of b-rHBsAg to monocytes. PBMC were incubated with b-rHBsAg in the presence of Ca²⁺ and Mg²⁺ (black bars) or Ca²⁺ and Mg²⁺ and 5 mM EDTA (grey bars) in 2% HS-50 mM Tris.HCl-150 mM NaCl, pH 7.4. After two washes with the same buffer cells were stained with Strep-PE, washed twice and analysed. Median fluorescence was determined. The data shown represent the average of two separate experiments. Error bars represent SD. Ca²⁺ and Mg²⁺ added after attachment of b-rHBsAg (white bars) does not reverse binding.

FIG. 5: Low pH prevents attachment of b-rHBsAg to monocytes. PBMC were incubated with b-rHBsAg in 2% HS-HBSS or 2% HS-10 mM citrate buffer-150 mM NaCl, pH 6 and 5 (black bars). After one wash step with the same buffer and one with 2% HS-HBSS, cells were stained with Strep-PE, washed twice and analysed. Median fluorescence was determined. The data shown represent the average of two separate experiments. Error bars represent SD. Pretreatment of PBMC (grey bars) or b-rHBsAg (white bars) with the same buffers does not prevent binding.

FIG. 6: mAb F47B reduces attachment of b-rHBsAg to monocytes and induces attachment to B lymphocytes.

b-rHBsAg was incubated with 5 μg/ml F47B. After 1 h PBMC were added and the cells were incubated for 90 minutes. After two washes cells were stained with Strep-PE, washed twice and analysed. Median fluorescence was determined. The data shown represent the average of three separate experiments with a total of n=6. Error bars represent SD.

FIG. 7: 1,25-dihydroxyvitamineD3 differentiated THP-1 cells express CD14 and bind b-rHBsAG

Non-differentiated THP-1 cells (white bars) or differentiated cells (black bars) were incubated with anti CD14-FITC clone P9, clone MY4 or b-rHBsAg in 2% HS-HBSS. After two washes with the same buffer b-rHBsAg was detected with Strep-PE. Cells were washed twice and analysed. Median fluorescence was determined. The data shown represent the average of two separate experiments. Error bars represent SD.

FIG. 8: b-rHBsAg attachment to PMA treated monocytes correlates with the expression level of My4 recognised CD14 molecules.

Non-treated cells (white bars) and PMA treated cells (black bars) were stained with anti CD14-FITC clone P9, clone MY4 or b-rHBsAg in 2% HS-HBSS. After two washes with the same buffer b-rHBsAg was detected with Strep-PE. Cells were washed twice and analysed. Median fluorescence was determined. The data shown represent the average of two separate experiments. Error bars represent SD.

FIG. 9: CD14 specific monoclonal antibodies block attachment of b-rHBsAg and b-HBsAg to monocytes of PBMC.

PBMC were incubated with different amounts of anti CD14 clone P9 (9a) or My4 (9b) in 2% HS-HBSS. After two washes with the same buffer PBMC were incubate with b-rHBsAg, washed twice and stained with Strep-PE. Cells were washed twice and analyzed, Median fluorescence was determined. The data shown represent the average of two separate experiments. Error bars represent SD. Left panel shows the effect of pre-incubation with anti CD14 mAbs on the attachment of b-rHBsAg; right panel shows the level of binding of these mAbs to monocytes. (c) Upper panel shows PBMC stained with SAPE only, the lower panels show staining of PBMC incubated with b-rHBsAg or b-HBsAg followed by SAPE. The interaction of b-HBsAg is blocked efficiently by mAb My4 and partially blocked by mAb P9 (right panels). This result is identical to the results obtained with b-rHBsAg (left panels).

FIG. 10: b-rHBsAg binds to CHO cells which express CD14 from cDNA.

CHO cells transfected with the empty plasmid (left panels) or cells expressing human CD14 (right panels) were incubated with anti CD14-FITC clone P9 (a, grey line), clone My4 (a, full line) or b-rHBsAg (b, full line) in 2% HS-HBSS. After two washes with the same buffer b-rHBsAg was detected with Strep-PE. Cells were washed twice and analysed. Dashed lines represent cells stained with isotypic controls or Strep-PE only.

FIG. 11: The S protein of rHBsAg particles is detected on CHO-CD14 cells by a serotype d specific monoclonal antibody.

a) CHO-CD14 cells were incubated for 90 minutes on ice with 5 or 25 μg/ml rHBsAg in 1% HS-HBSS. After 2 washes cells were incubated for 1 h with 5 μg/ml of different mouse mAbs as indicated. After 2 washes mAbs were detected with rabbit anti-mouse-FITC. Cells were washed twice and analyzed. b) CHO-CD14 and control CHO cells were incubated with 5 or 25 μg/ml rHBsAg in 1% HS-HBSS. After 2 washes cells were incubated for 1 h with 5 μg/ml of mouse anti-d. After 2 washes the mAb was detected with rabbit anti-mouse-FITC. Cells were washed twice and analysed.

FIG. 12: Binding of b-rHBsAg to monocytes is blocked by mAbs My4, biG4 and biG11 which bind (close) to the LPS binding site of CD14.

PBMC were incubated with 0.25 μg/ml (grey bars), 1 μg/ml (white bars) and 2.5 μg/ml (black bars) of different CD14 specific antibodies and 2.5 μg/ml of isotype controls in 2% HS-HBSS. After 1 h of incubation on ice b-rHBsAg was added and the cells were incubated for another 80 minutes. After 2 washes b-rHBsAg was detected with Strep-PE. Cells were washed twice and analysed. Median fluorescence was determined. The data shown represents the average of two separate experiments. Error bars represent SD.

FIG. 13: Attachment of b-rHBsAg to monocytes is enhanced by low concentrations of serum and reduced at higher concentrations.

PBMC were incubated for 80 minutes on ice with b-rHBsAg in HBSS containing different concentrations of human AB serum or BSA. After 2 washes b-rHBsAg was detected with Strep-PE. Cells were washed twice and analysed. Median fluorescence was determined. The data shown represents the average of three separate experiments. Error bars represent SD.

FIG. 14: Attachment of b-rHBsAg to CHO-CD14 cells is enhanced by low concentrations of serum.

CHO cells transfected with the empty plasmid or cells expressing CD14 were incubated with b-rHBsAg with (white bars) or without (black bars) 2% HS in HBSS. After 2 washes b-rHBsAg was detected with Strep-PE. Cells were washed twice and analysed. Median fluorescence was determined. The effect of 2% serum on PBMC were performed as a control.

FIG. 15: Heat inactivation of serum destroys the factor that enhances binding of b-rHBsAg to monocytes.

PBMC were incubated for 80 minutes on ice with b-rHBsAg in HBSS without serum (white bar), with 2% HS (black bar), with 2% HS which was heat inactivated for 30 minutes (dark gray bar) or 60 minutes (light grey bar). After 2 washes b-rHBsAg was detected with Strep-PE. Cells were washed twice and analysed. Median fluorescence was determined. The data shown represents the average of three separate experiments. Error bars represent SD.

FIG. 16: Soluble CD14 inhibits attachment of b-rHBsAg to monocytes.

b-rHBsAg was incubated on ice with different amounts of recombinant soluble CD14 in 2% HS-HBSS. After 1 h PBMC were added and incubated on ice for 80 minutes. After 2 washes b-rHBsAg was detected with Strep-PE. Cells were washed twice and analyzed. Median fluorescence was determined. The data shown represents the average of three separate experiments. Error bars represent SD.

FIG. 17: Purified recombinant LBP enhances binding of b-rHBsAg to monocytes. PBMC were incubated on ice for 80 minutes with b-rHBsAg in HBSS with or without 2% HS or with different concentrations of purified recombinant LBP. After 2 washes b-rHBsAg was detected with Strep-PE. Cells were washed twice and analysed. Median fluorescence was determined. The data shown represents the average of two separate experiments. Error bars represent SD.

FIG. 18: LBP specific mAbs inhibit attachment of b-rHBsAg to monocytes. PBMC were incubated on ice for 80 minutes with b-rHBsAg and 0.25 μg/ml (white bars) or 2 μg/ml (black bars) of two different LBP specific antibodies or 2.5 μg/ml of isotype control in 2% HS-HBSS. After 2 washes b-rHBsAg was detected with Strep-PE. Cells were washed twice and analyzed. Median fluorescence was determined. The data shown represents the average of two separate experiments. Error bars represent SD.

FIG. 19: rHbsAg can inhibit attachment of CD14 specific monoclonal antibodies PBMC were incubated with 50 μg/ml rHBsAg and 0.5 μg/ml LBP in 1% HS-HBSS. After 90 minutes, cells were washed and incubated with 1 μg/ml of the different CD14 specific mAbs or b-rHBsAg. Antibodies were detected with goat anti-mouse-FITC, b-rHBsAg was detected with SAPE. The left panel shows the median fluorescence measured; the right panel shows the % inhibition. The data shown represents the average of three separate experiments. Error bars represent SD.

FIG. 20: Phosphatidyl-serine and phosphatidyl-glycemol block the binding of b-rHBsAg to monocytes of PBMC.

PBMC were incubated with 10 (black bars) or 40 (white bars) μg/ml of PC, PS and DOPG in 1% HS-HBSS. After washings b-rHBsAg was added and the cells were incubated for another 80 minutes. After 2 washes b-rHBsAg was detected with StrepPE. Cells were washed twice and analysed. Median fluorescence was determined. The data shown represents the average of three separate experiments. Error bars represent SD.

FIG. 21: Phosphatidylserine and phosphatidylglyceml reconstituted HBsAg particles block the binding of brHBsAg to monocytes of PBMC.

PBMC were incubated with 10 (black bars) or 50 (white bars) μg/ml of different HBsAg preparations in 1% HS-HBSS containing 0.5 μg/ml LBP. After washings b-rHBsAg was added and the cells were incubated for another 80 minutes. After 2 washes b-rHBsAg was detected with Strep-PE. Cells were washed twice and analysed. Median fluorescence was determined. The data shown represents the average of three separate experiments. Error bars represent SD.

FIG. 22: Antigenicity of delipidated and reconstituted particles.

Elisa plates were coated with 0.5 μg/ml of rHBsAg, HBsAg, delipidated HBsAg, PC-HBsAg, PS-HBsAg DOPG-HBsAg. After blocking with BSA, human mAbs F47B, F9H9, anti-a and the mouse mAb antid-d were allowed to bind to the particles. mAbs were detected with either goat-anti-mouse or goat-anti-human-peroxidase labeled antibodies.

FIG. 23: rHBsAg reduces LPS induced IL-1β and TNFα secretion.

1,25-dihydroxyvitamineD3 differentiated THP-1 cells (a) or PBMC from donor 1 (black symbols) and donor 2 (white symbols) (b) were incubated with 0 (♦), 10 (570 ) or 50 (▴) ng/ml LPS and different concentrations of rHBsAg. Cell supernatant was collected after 24 h and cytokine concentrations determined.

FIG. 24: rHBsAg reduces LPS induced IL-1β and TNFα secretion, but enhances IL-10 secretion.

PBMC from donor 1 (black symbols) and donor 2 (white symbols) were incubated with 0 (not shown because below detection limit) or 10 ng/ml (□,▪) LPS and different concentrations of rHBsAg. Cell supernatant was collected after 24 h and cytokine concentrations determined.

### EXAMPLES

### Example 1

Materials and Methods

HBsAg Preparations.

Purified rHBsAg (subtype adw₂) produced in S. cerevisiae [(lots DVP23 (752 μg/ml in PBS), DVP 93/1 (1 mg/ml) and DVP 93/2 (1 mg/ml)] were from SmithKline Beecham Biologicals, Rixensart, Belgium. The purity of these S-derived rHBsAg preparations is judged by HPLC analysis as well as SDS-PAGE with Coomassie staining and is >98%. These rHBsAg preparations are used normally as HBV vaccines for humans after adsorption on to aluminium hydroxide.

Plasma purified HBsAg (0.28 mg/ml), delipidated HBsAg (0.3 mg/ml), PC, PS and DOPG-reconstituted HBsAg (0.3 mg/ml, 0.16 mg/ml and 0.3 mg/ml, respectively) as well as the lipids PC (1.85 mg/ml), PS (1.5 mg/ml and DOPG (1.9 mg/ml) were provided by Francisco Gavilanes and Darell Peterson.

Biotinylation of HBsAg

HBsAg or rHBsAg was biotinylated using an ECL protein biotinylation module (RPN 2202, Amersham Pharmacia Biotech). 300 μI HBsAg was mixed with 270 μl H₂O, 30 μl 0.8 M bicarbonate buffer pH 8.6 and 15 μl biotinylation reagent. The mixture was incubated at RT for 1 h, after which 24 μl 1 M Tris was added. Biotinylated HBsAg was purified by gel filtration on a Sephadex G25 column using PBS. 1 ml fractions were collected and b-(r)HBsAg peak fractions were determined by ELISA.

sCD14 and LBP

sCD14 and LPS binding protein (LBP) were from Dr. Felix Stelter (Stelter et al., 1997).

Antibodies

Mouse anti-human CD3-FITC (clone SK 7), CD14 and CD14-FITC (clone M(P9), CD19-FITC (clone 4G7) and Streptavidine-phycoerythrin (Strep-PE) were from Becton Dickinson. Mouse anti-human CD14 and CD14-FITC (clone My4) were from Immunotech. Mouse anti-human CD18-FITC (clone 6.7) was from Pharmingen. Ascitic fluid of mouse anti-human CD14 clones biG4 (IgG1) and biG11 (IgG1) were from Dr. Felix Stelter (Stelter et al., 1997). Mouse anti-human CD14 clone biG2 (IgG2a) and mouse anti-human LBP clones biG48 (IgG1), biG412 (IgG1) and rabbit anti-human CD14 were from Biometec (Greifswald, Germany). Rat anti-mouse CD14-FITC (clone rmC5-3, IgG1) was from Pharmingen. Mouse anti-HBsAg monoclonal antibodies, specific for serotypes d and y, were from DiaSorin. Human anti-a was developed in the Center for Vaccinology (Gent) Human anti-HBsAg clones F47B and F9H9 were from Lia Sillekens (Centraal laboratorium van de bloedbank, Amsterdam).

The following isotypic controls were used: mouse IgG1 (Becton Dickinson) or ascitic fluid of LMBH6 (Vanlandschoot et al., 1998) and IgG1-FITC (Becton Dickinson), mouse IgG2a (Pharmingen), mouse IgG2a-FITC (Caltag), mouse IgG2b and IgG2b-FITC (Coulter), rat IgG1-FITC (Pharmingen) and rabbit IgG (Sigma).

Non labelled antibodies were detected with goat anti-rabbit IgG-FITC (Pharmingen) and rabbit anti-mouse F(ab′)2-FITC (DAKO).

ELISA

Maxisorp 96-well plates (Nunc, Roskilde, Denmark) were coated with B-rHBsAg in PBS. The wells were blocked with 0.1% BSA in PBS, followed by three washings (0.1% Triton X-100). Streptavidin-horse radish peroxidase (1/1000 in PBS-0.1% BSA) was added and the plates were incubated for 1 h at room temperature. After three washings 3.3′,5,5′-tetramethylbenzidine (Sigma) was added. After 30 minutes the reaction was stopped with 0.1 N H₂SO_{4.}

Cells

Human PBMC were isolated from buffy coats using Ficoll-Hypaque (density=1.077 g/ml, Nycomed Pharma, Oslo, Norway) centrifugation. Cells were stored in liquid nitrogen. Phorbol-12-myristate-13-acetate treatment (25 ng/ml) (PMA, Sigma) was performed for 4 h in cRPMI (RPMI 1640-10% FCS-2 mM L-glutamine-1 mM Na-pyruvate-50 U/ml penicilline-50 μg/ml streptomycine-20 μM β-mercaptoethanol). THP-1 cells were grown in complete cRPMI. To induce differentiation 100 nM 1,25-dihydroxyvitamin D3 (1,25-VitD3, Calbiochem) was added for 48 h. CHO cells expressing human CD14, CD14mut6, mouse CD14 and CHO cells transfected with the vector only (Jack et aL, 1995) were grown in MEM alpha without nucleosides and ribonucleosides (Gibco-BRL) supplemented with 10% FCS, 2mM L-glutamine-50 U/ml penicilline-50 μg/ml streptomycine-100 nM (plasmid transfected CHO cells) or 500 nM methotrexate (CD14 expressing CHO cells). Cultured cells were detached mechanically or by using non enzymatic cell dissociation buffer (Sigma), washed twice with 2% HS-HBSS and stained as described below.

Staining of Cells

PBMC were thawed and washed twice with 1-2% HS-HBSS. 10⁶ cells were incubated with b-rHBsAg in 200 μl 2% HS-HBSS for 1 h on ice. After 2 washes with the same solution, cells were incubated with Strep-PE and/or FITC labelled antibodies in 2% HS-HBSS for 1 h on ice. After 2 washes cells were resuspended in 1 ml 2% HS-HBSS or PBS, containing propidium iodide (PI) and analysed on a FACScan flow cytometer (Becton Dickinson). Dead cells which incorporated PI were gated out of analysis. At least 5000 cells were counted per analysis. Fluorescence (530 nm for FITC and 580 nm for PE) was measured. Median fluorescence was determined in each case. The signals were acquired in a logarithmic mode for Fl1 (FITC) and Fl2 (PE). Treshhold levels were set according to negative (Strep-PE only) and isotypic controls. Gates for monocytes were set to the position in the SSC and the FSC.

LPS Treatment of THP-1 and PBMC.

5.10⁵ THP-1 cells were treated for 24 h with 100 nM 1,25-dihydroxyvitamin D3 (1,25-VitD3, Calbiochem). After washing, the cells were incubated in cRPMI with or without 10 or 50 ng/ml LPS (E. coli 0111:B4, Sigma). 0, 0.1, 1, 10 or 50 μg/ml rHBsAg was added. 10⁶ PBMC were incubated in cRPMI with or without 10 or 50 ng/ml LPS. 0, 0.1, 1, 10 or 25 μg/ml rHBsAg was added. Cell supernantants were collected after 24 h and tested for the presence of IL-1β, IL-10 and TNFα.

IL-2 Treatment of PBMC.

10⁶ PBMC were incubated in cRPMI with or without 1000 U/ml IL-2. 0, 1, 10, 25 or 50 μg/ml rHBsAg was added. Cell supernantants were collected after 24 h and tested for the presence of IL-8.

Cytokine Determinations.

The concentration of IL-1β, IL-8, IL-10 and TNFα in cell supernatant was determined using commercially availble kits (Bioscource) according to the manufactors instructions.

### Example 2

Effect of Biotinylation on the Antigenicity of rHBsAg.

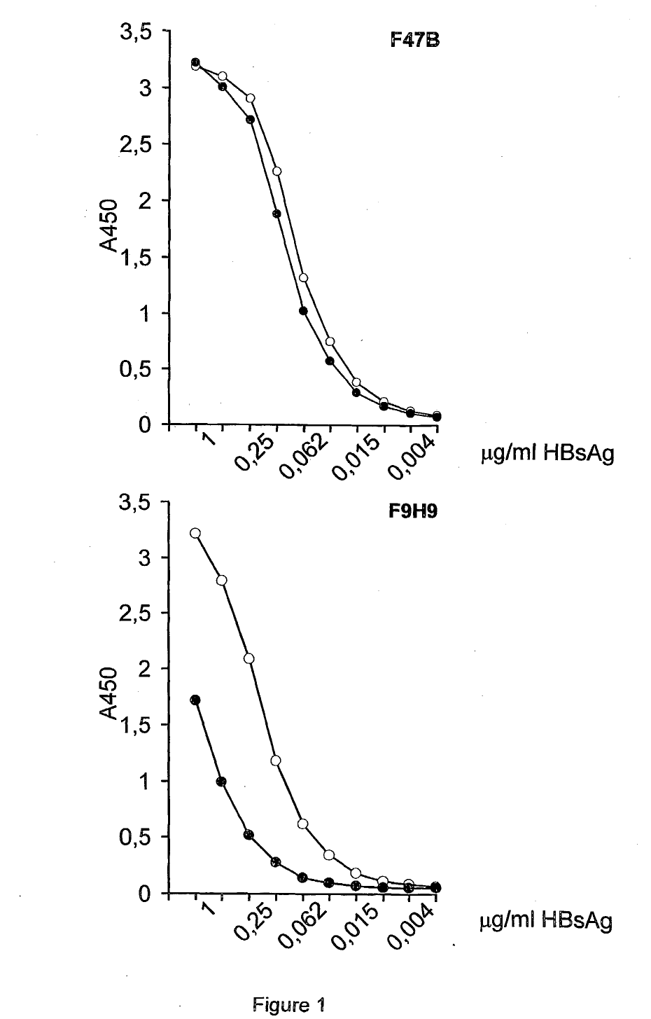

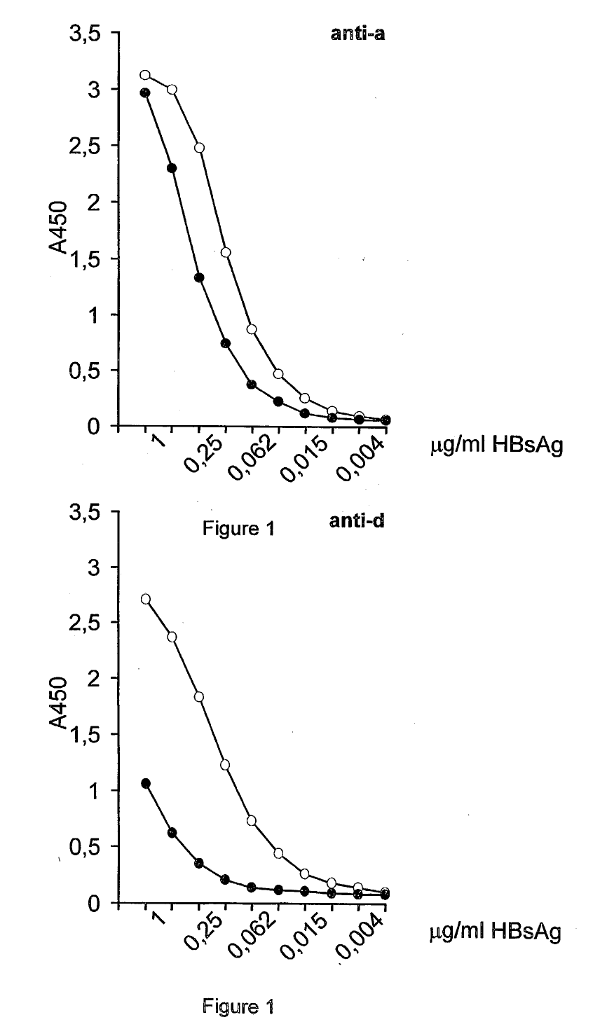

rHBsAg was biotinylated and purified as described. Two b-rHBsAg peak fractions were determined by ELISA. b-rHBsAg was still recognized in a HBsAg specific radioimmunoassay (AUSRIA Abott Laboratories, Chicago Ill.). Because the three lysine residues which can become biotinylated all lie in the major antigenic region of the S protein, the reactivity of b-rHBsAg towards 4 HBs-Ag-specific mAbs was investigated (FIG. 1). Biotinylation did not influence the binding of mAb F47B (Stricker et al., 1985) which recognizes an epitope in the carboxy terminal end of the S protein (Paulij et al., 1999), a region which was initially predicted to form a membrane spanning domain (Stirck et al., 1992). Binding of mAb anti-d was reduced strongly after biotinylation. This is not unexpected as the lys residues at positions 122 is the key determinant for the ‘d’ and ‘w’ serotypes, respectively. Binding of the mAbs F9H9 and anti-a was reduced strongly or reduced only slightly, respectively. This diminished reactivity is probably due to biotinylation at residue 141, which lies within the ‘a’ determinant.

### Example 3

Characterization of b-rHBsAg Binding to PBMC.

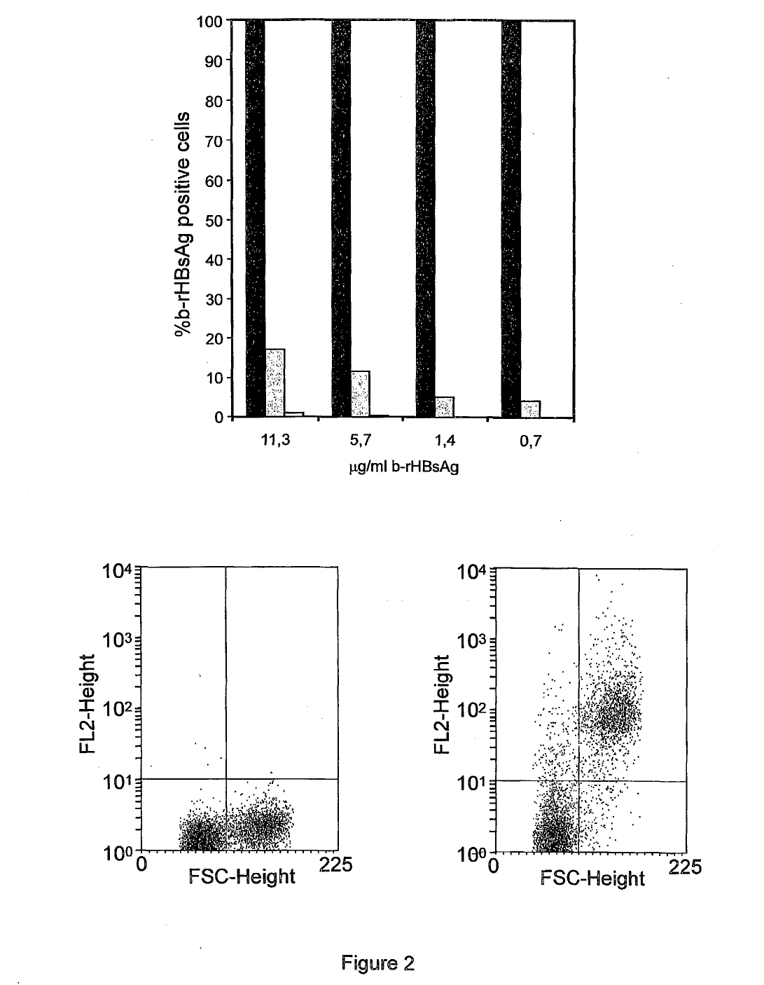

PBMC were incubated with different concentrations of b-rHBsAg after which the cells were incubated with mAbs specific for monocytes, T or B-cells. b-rHBsAg was detected with Strep-PE. At the lowest b-rHBsAg concentration almost only CD14+ cells were b-rHBsAg positive. At higher concentrations a fraction of CD19+ cells became positive for b-rHBsAg as well. Binding of b-rHBsAg to CD3+ cells was never detected (FIG. 2). Based on these results it was decided to use approximately 1-2 μg/ml b-rHBsAg in all experiments. Although biotinylation clearly altered the attachment of several mAbs, it did not prevent the interaction with the monocytes of PBMC. This results suggest that amino acid residues of the major antigenic region are not important for the interaction with the receptor on monocytes.

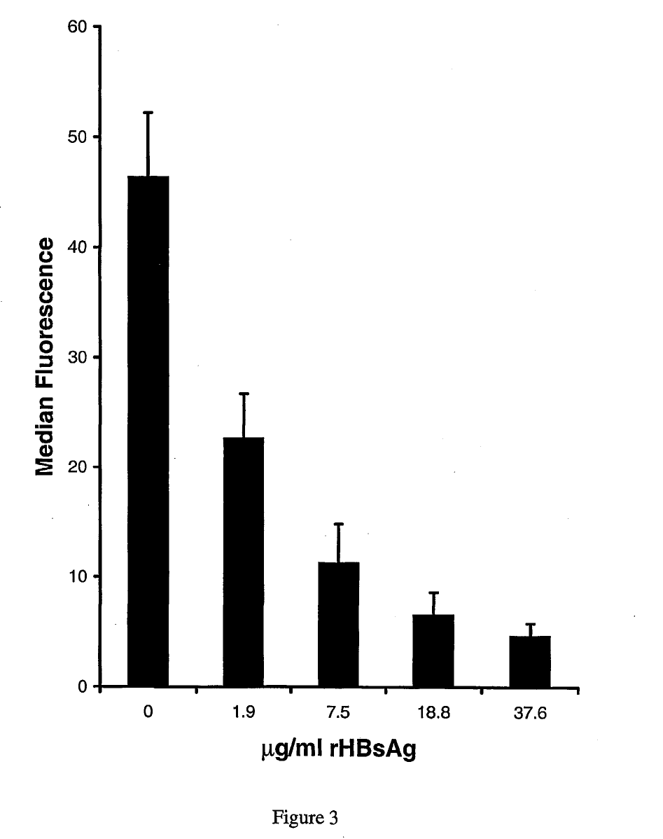

To demonstrate the specificty of the observed interaction, PBMC were incubated with different amounts of non-biotinylated rHBsAg. After 1 h b-rHBsAg was added to this mixture. As shown in FIG. 3, unlabelled rHBsAg blocked the binding of b-rHBsAg. Inhibition was also observed when non-biotinylated rHBsAg from lot DVP93/1 and DVP93/2 were used (data not shown).

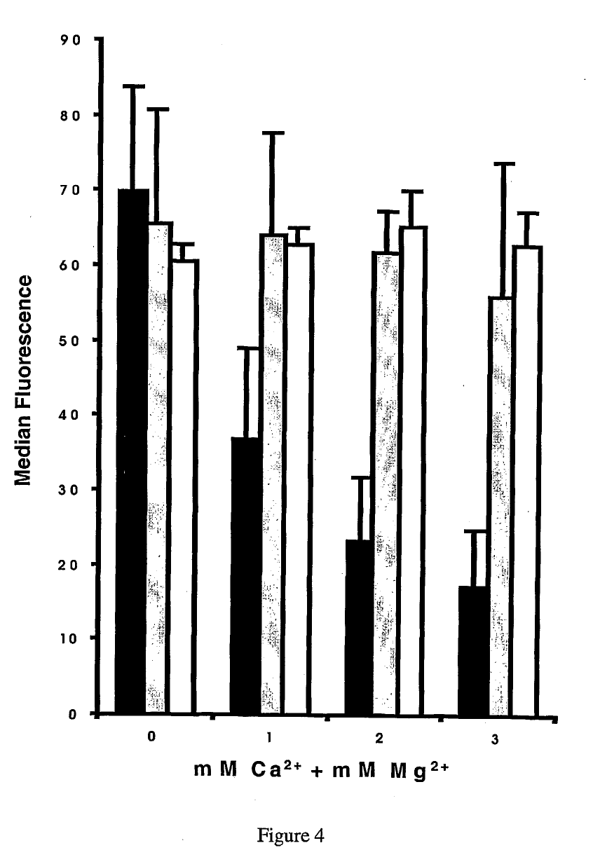

The interaction of HBV proteins with cells or putative cellular receptors has often been claimed to be Ca²⁺ dependent or independent. (Hertogs et al., 1993; Neurath et al., 1990; Komai and Peeples, 1990; Mehdi et al., 1996). Contrary to these reports, the addition of increasing amounts of Ca²⁺ and Mg²⁺ caused reduced binding of b-rHBsAg. The addition of 5 mM EDTA to the mixture restored attachment. The addition of Ca²⁺ and Mg²⁺ after binding had no effect (FIG. 4). Reduced binding was also observed when only Ca²⁺ or only Mg²⁺ was added (results not shown). These experiments were all performed in 2% HS-50 mM Tris.HCl-150 mM NaCl instead of HBSS to prevent acidification when adding 5 mM EDTA.

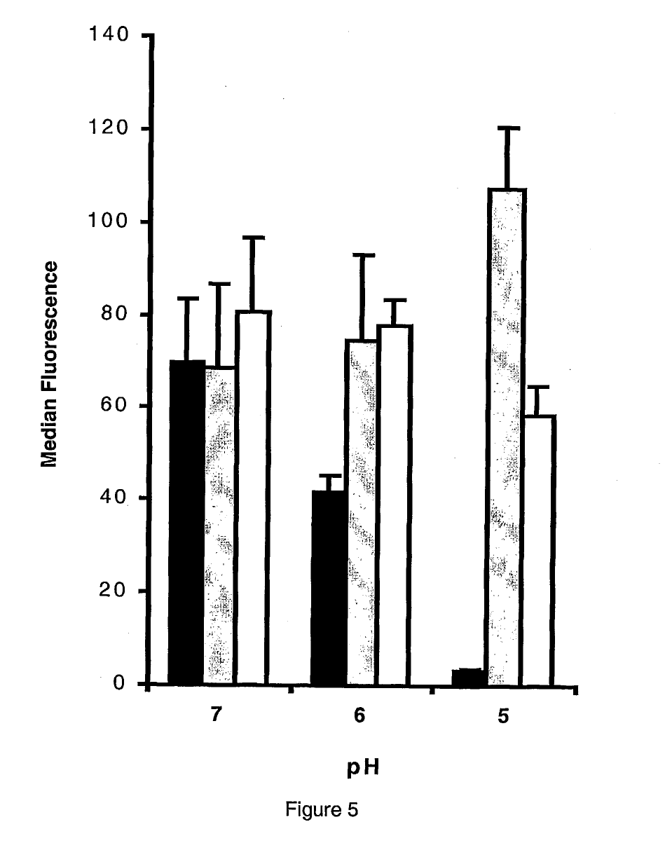

Conflicting results about the influence of low pH on virus infection or HBsAg attachment have been reported. (Komai and Peeples, 1990; Mehdi et al., 1996; Lu et al., 1996; Hagelstein et al., 1997). Therefor the effect of pH on particle binding was measured at pH 7, 6 and 5. As shown in FIG. 5 b-rHBsAg binding was strongly reduced when the pH was lowered. Pre-incubation of the cells or b-rHBsAg at the same pH and for the same time did not have any effect.

### Example 4

mAb F74B Inhibits Attatchment of b-rHBsAg to Monocytes.

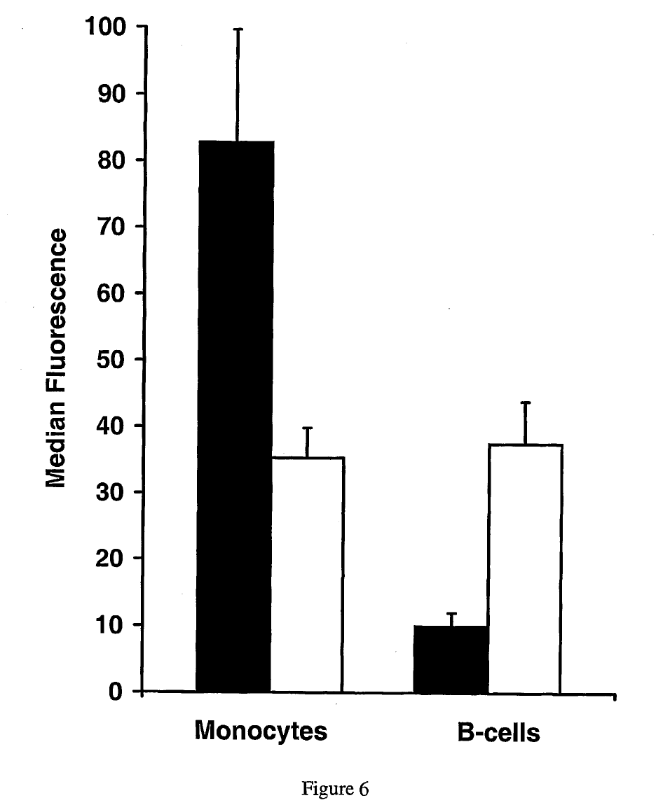

If the binding of b-rHBsAg to the monocytes is specific, antibodies to HBsAg should be able to block the interaction. To test this prediction b-rHBsAg was incubated with different concentrations of F47B, F9H9, anti-a and anti-d. Only with mAb F47B a dose dependent reduction in binding was observed (data not shown). This was not unexpected because biotinylation interferes with the binding of the other mAbs to rHBsAg. Surprisingly with mAb F47B a dose dependent binding of b-rHBsAg to 20-35% of the lymphocytes was detected, which were identified as B-lymphocytes (not shown). Maximal inhibition of b-rHBsAg rHBsAg binding to the monocytes and maximal attachment to the B-cells was obtained already with 5 μg/ml F47B (FIG. 6).

### Example 5

Effect of the Monocyte Differentiation State on the Attachment of b-rHBsAG.

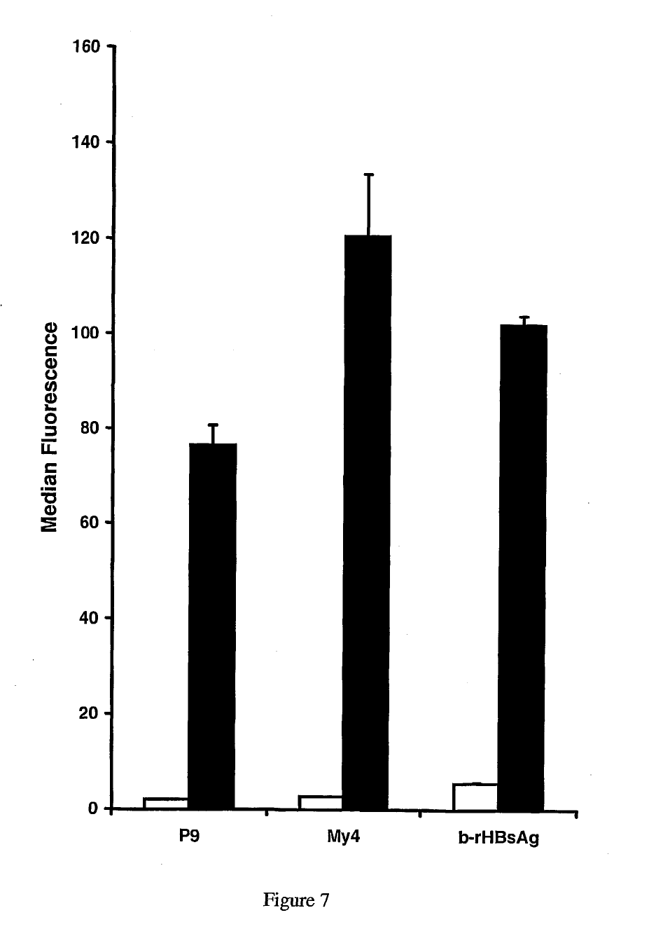

THP-1 cells, a pre-monocytic cell line which does not express CD14, differentiates towards a monocytic cell type by treatment with 1,25 dihydroxyVitaminD3. This differentiation is easily detected by the expression of CD14. Binding of b-rHBsAg to this pre-monocytic cell line and 1,25-VitD3 differentiated THP-1 was investigated. CD14 expression was detected using two different specific mAbs, clone P9 and My4. Both antibodies were used because they recognize different forms of CD14, which may differ in expression levels on monocytes and monocytic cell lines (Pedron et al., 1995). Undifferentiated THP-1 cells showed no detectable expression of CD14 and did not bind b-rHBsAg. 1,25-VitD3differentiated THP-1 cells expressed CD14 molecules which were recognized by both antibodies. These differentiated cells did bind b-rHBsAg (FIG. 7).

### Example 6

Effect of CD14 Expression Levels on the Attachment of b-rHBsAq to Monocytes.

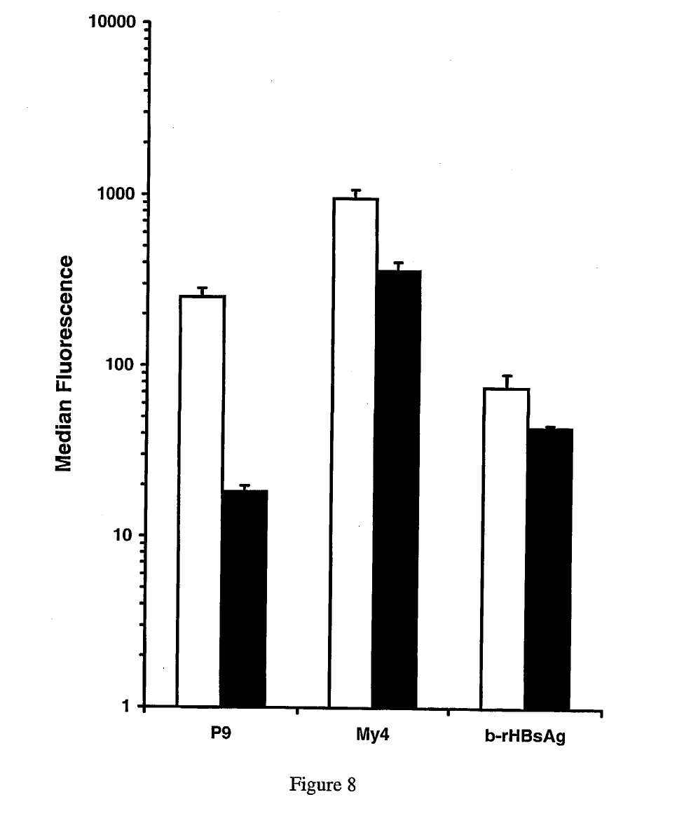

The effect of PMA treatment on b-rHBsAg binding to PBMC was examined. An established feature of CD14 is that monocytes shed it rapidly when treated with PMA (Bazil and Strominger, 1991). Again anti-CD14 antibodies, clone P9 and My4, were used to detect changes in CD14 levels. Similar to previous reports (Pedron et al., 1995) PMA treatment removed CD14 molecules recognized by clone P9 almost completely, while CD14 molecules recognized by clone My4, although reduced, showed still a high expression level (FIG. 8). b-rHBsAg attachment to the PMA treated monocytes was only slightly reduced. These results suggest that the presence of My4 recognized CD14 molecules might be important for attachment of b-rHBsAg.

### Example 7

Effect of CD14 Specific Antibodies on b-rHBsAg Binding.

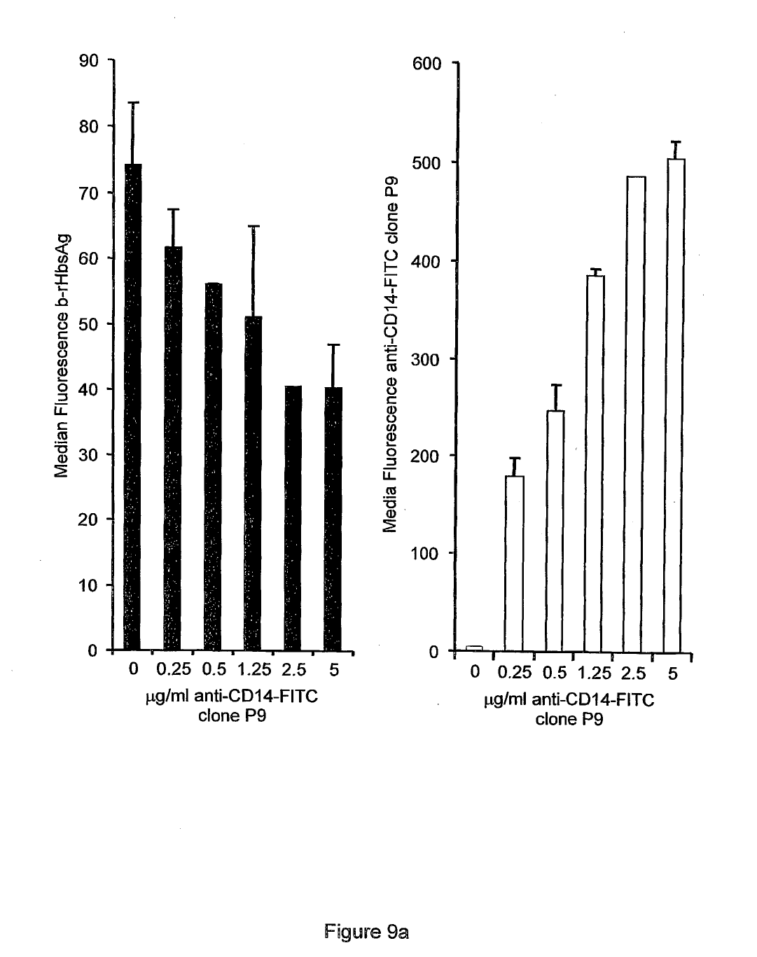

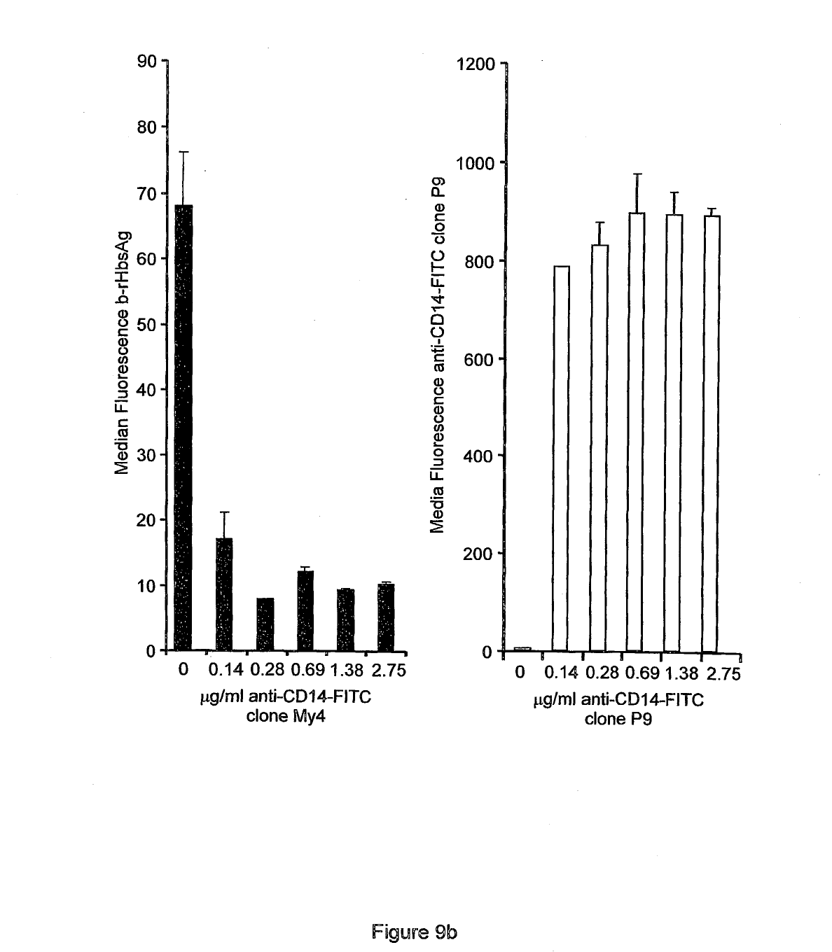

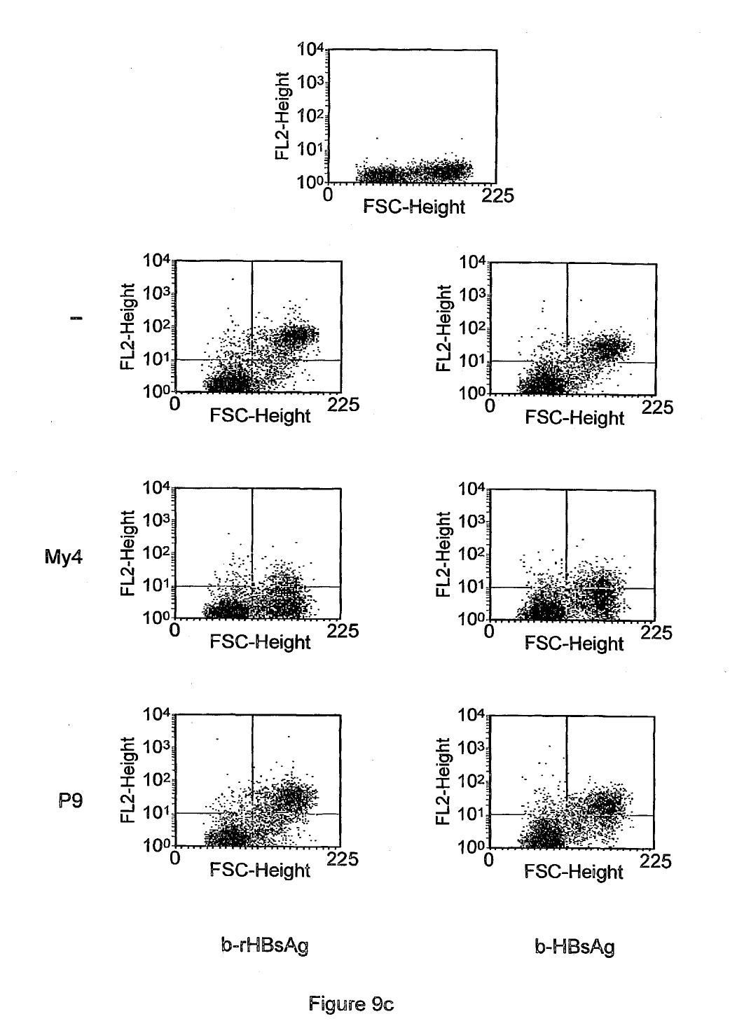

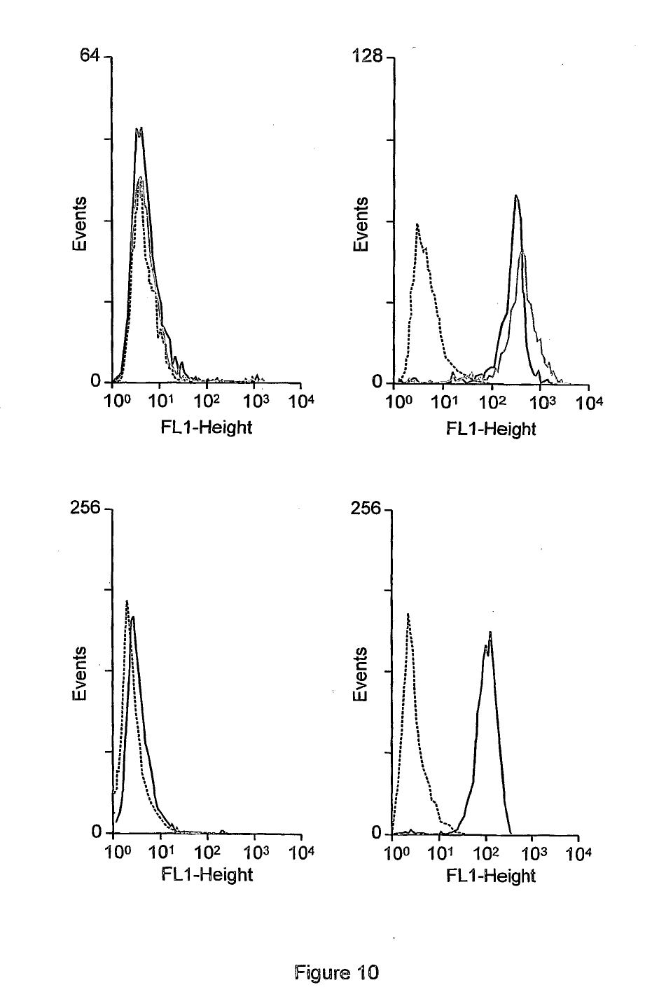

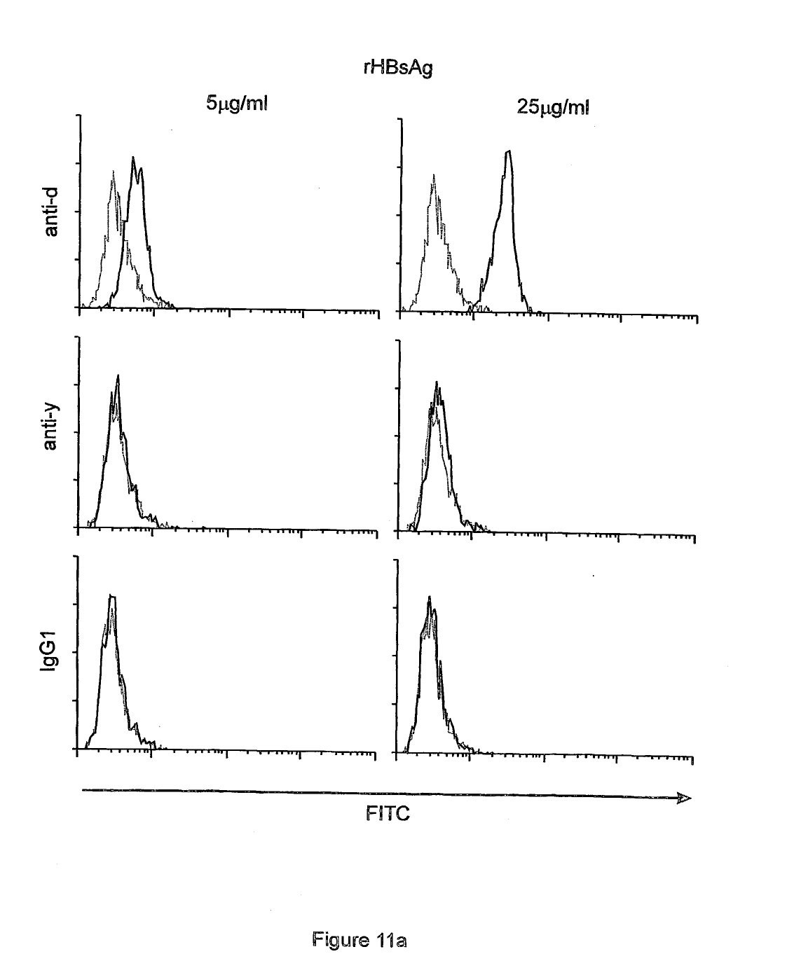

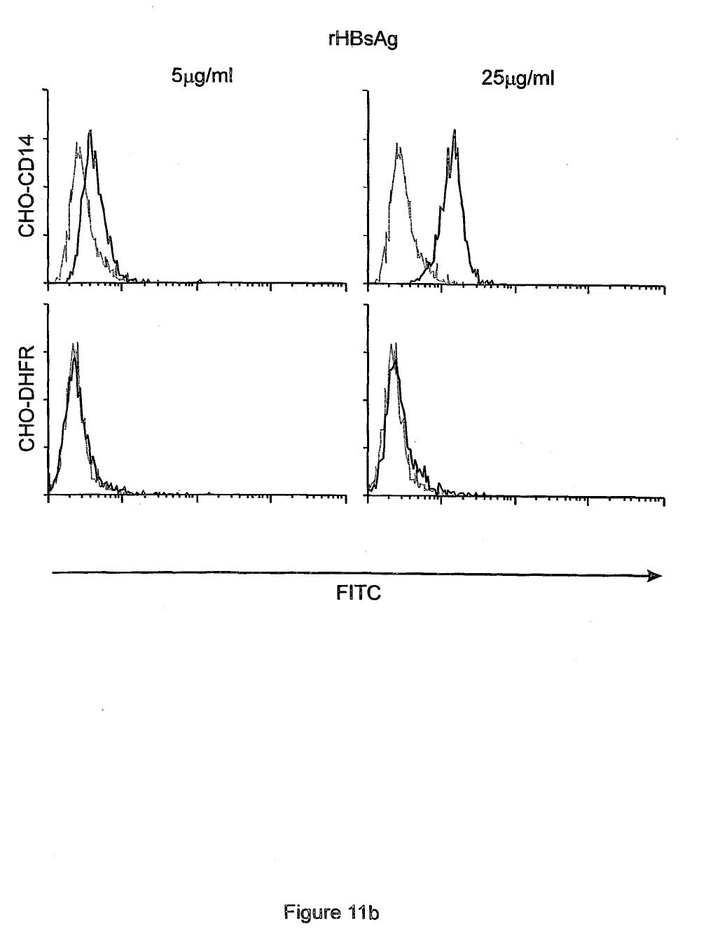

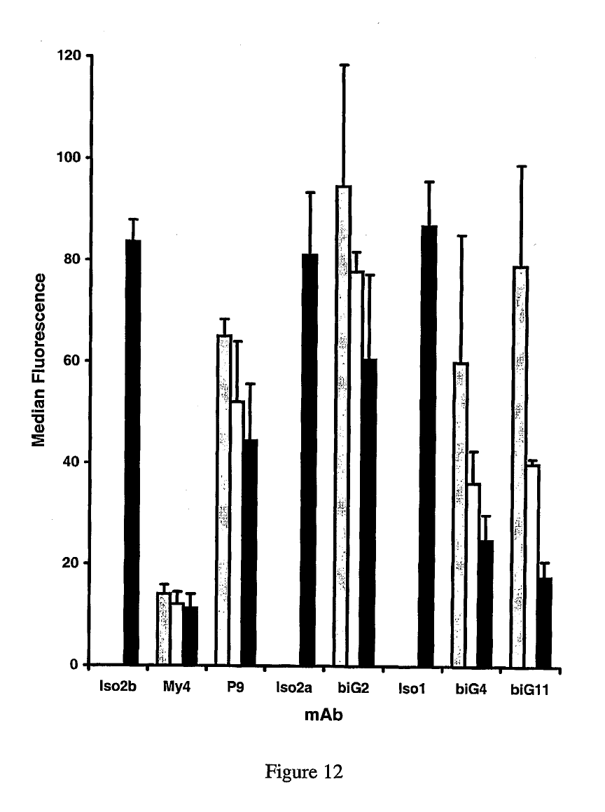

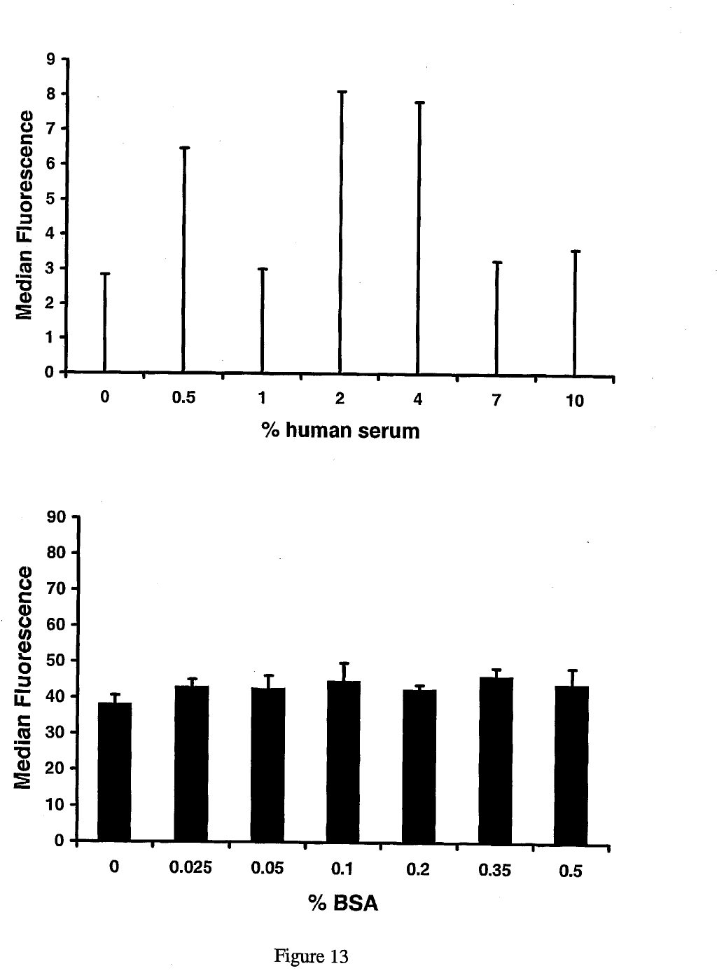

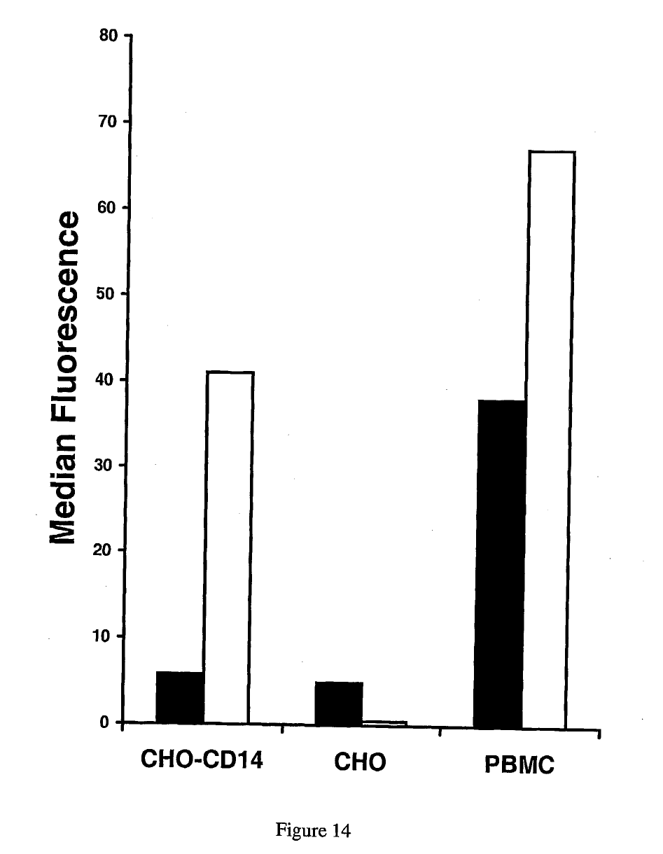

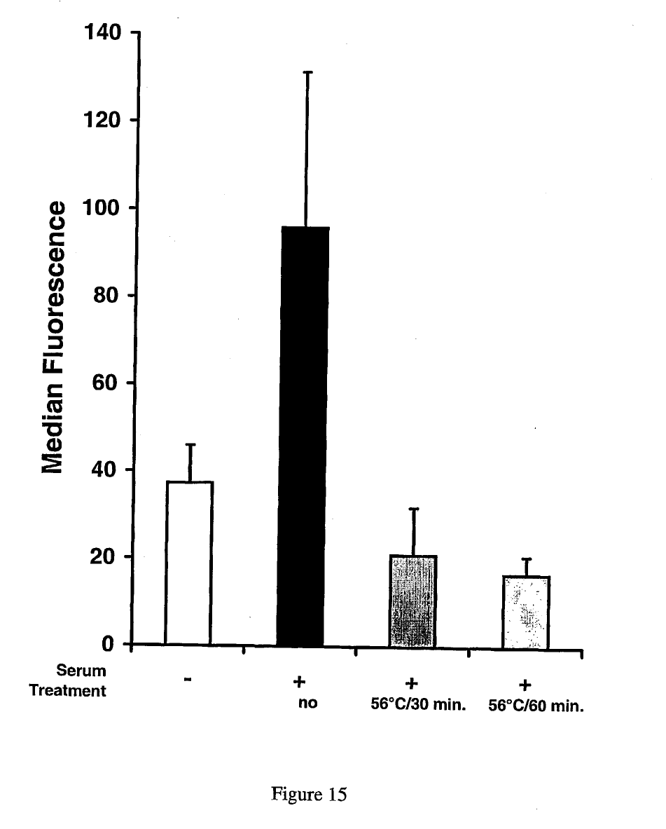

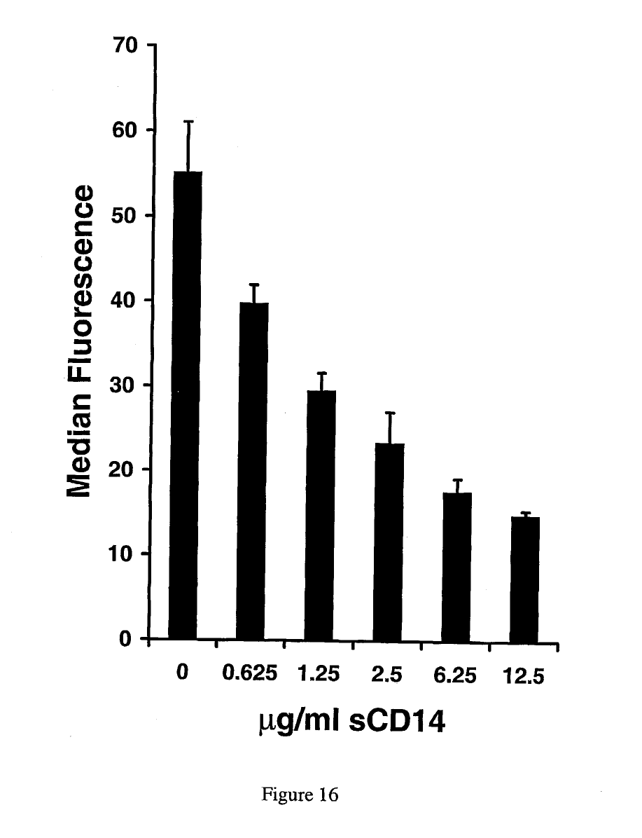

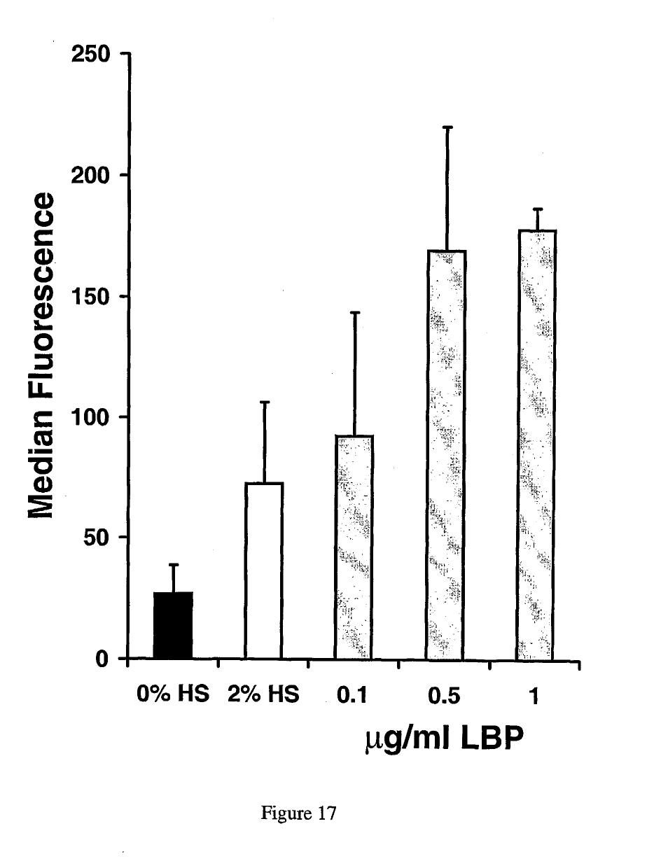

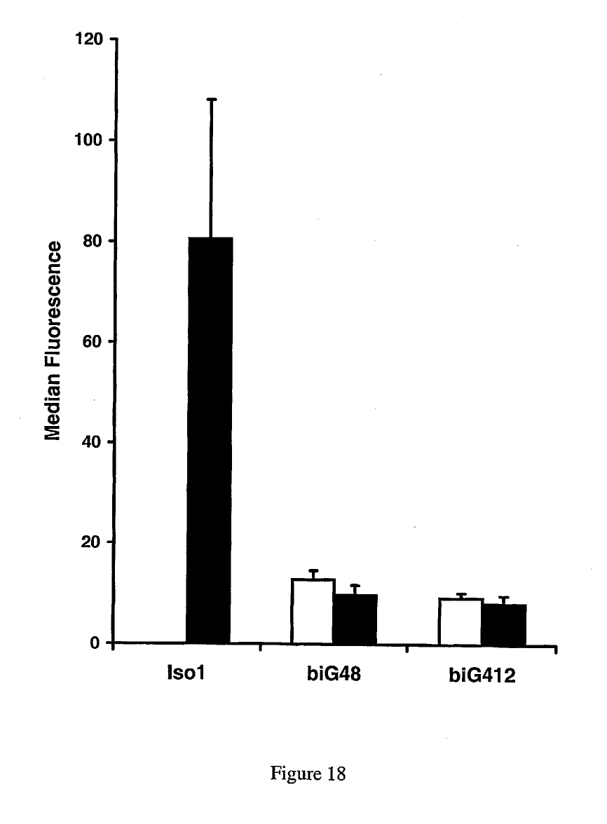

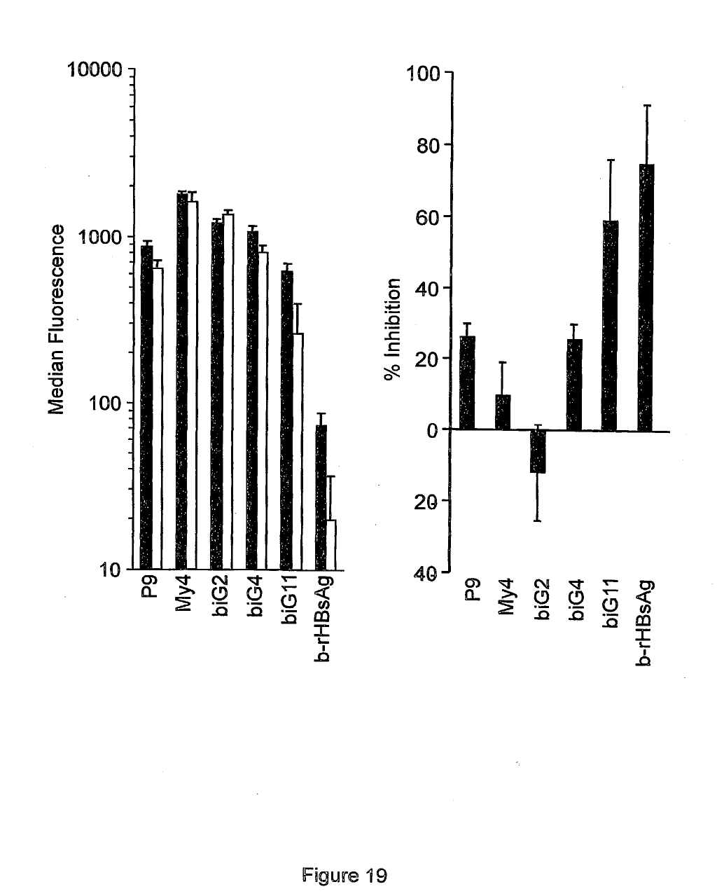

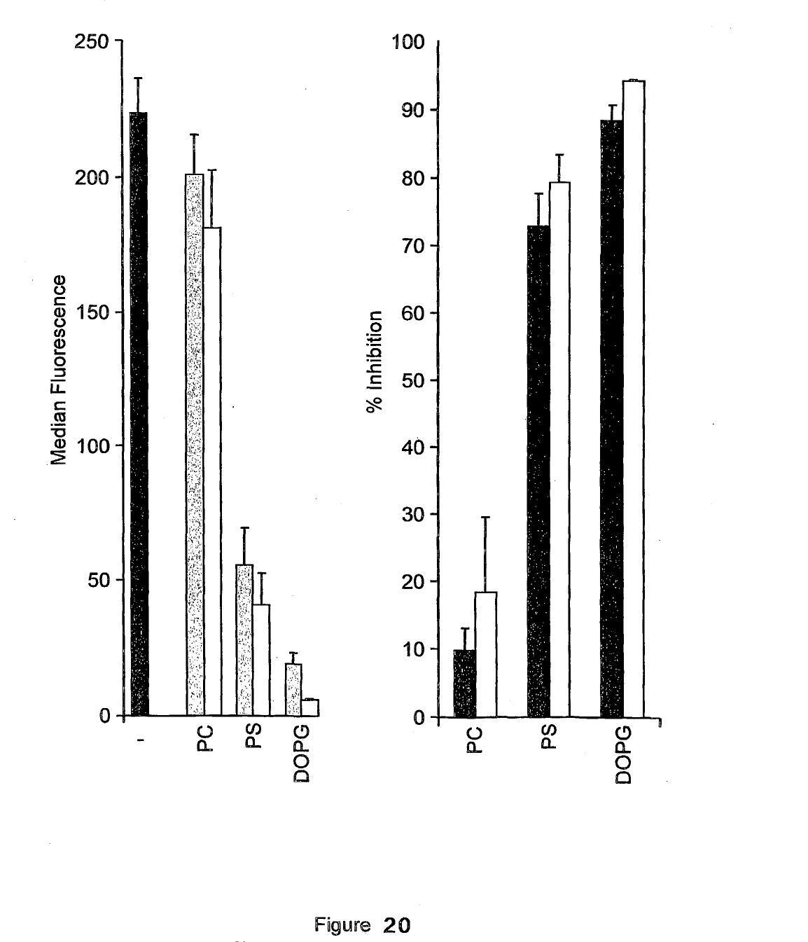

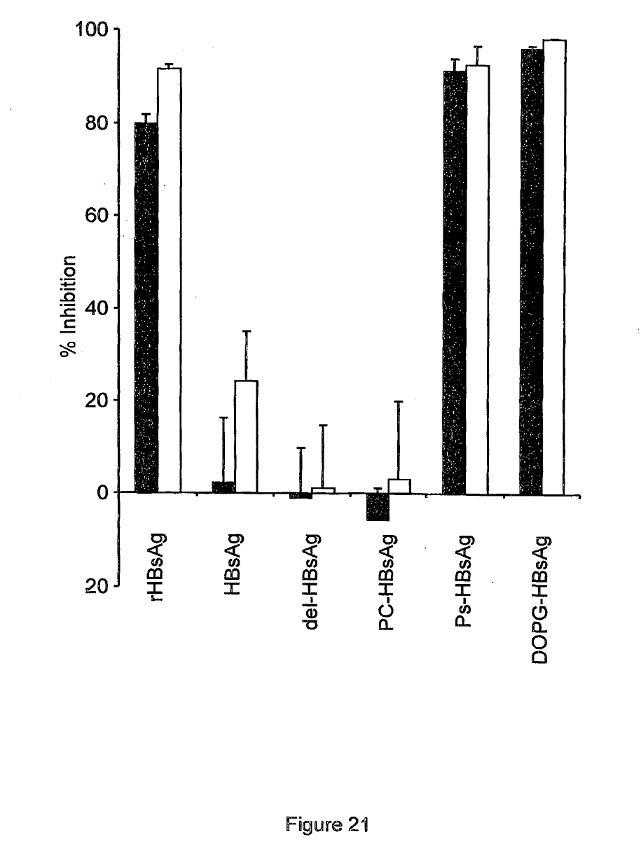

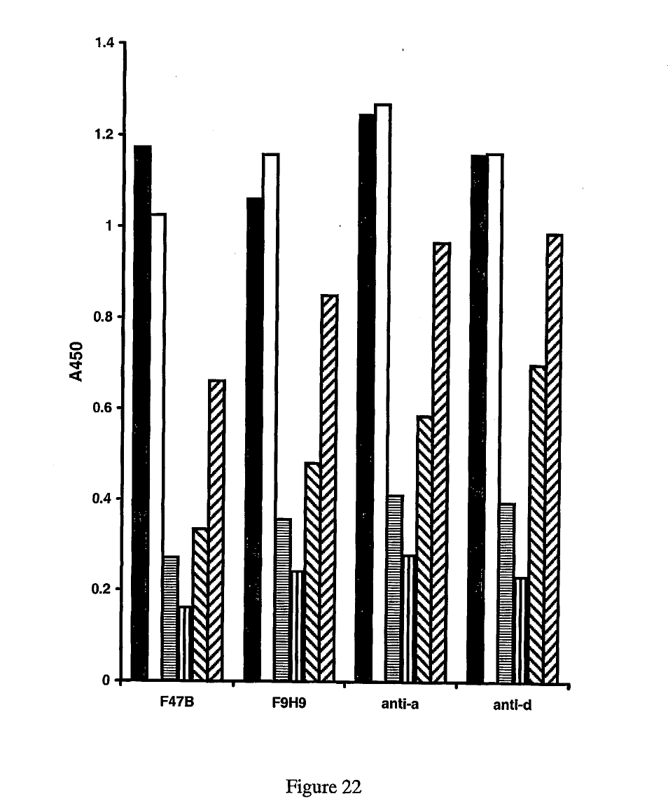

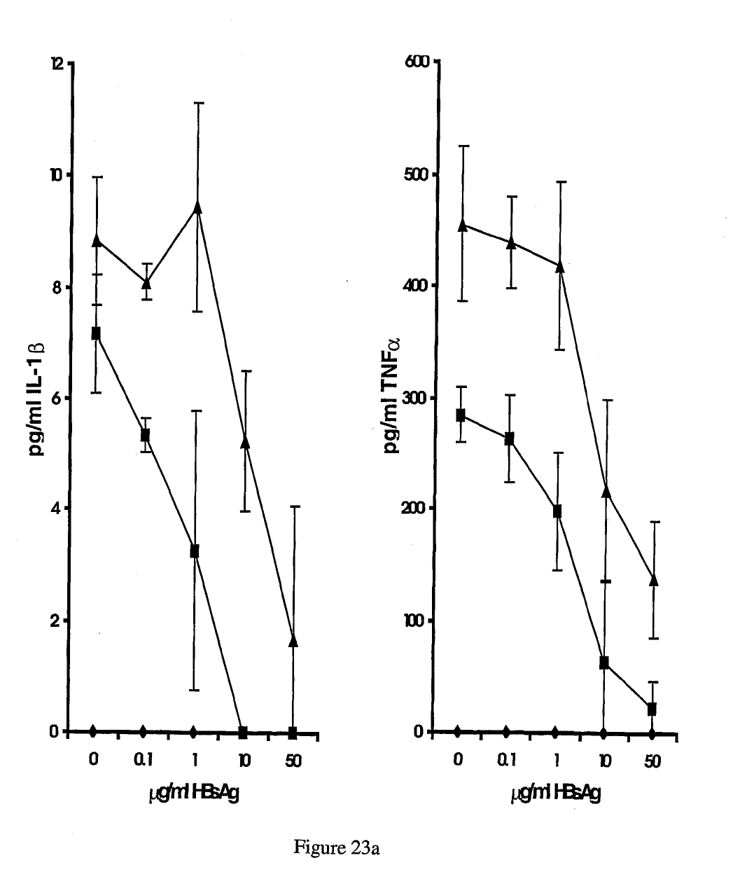

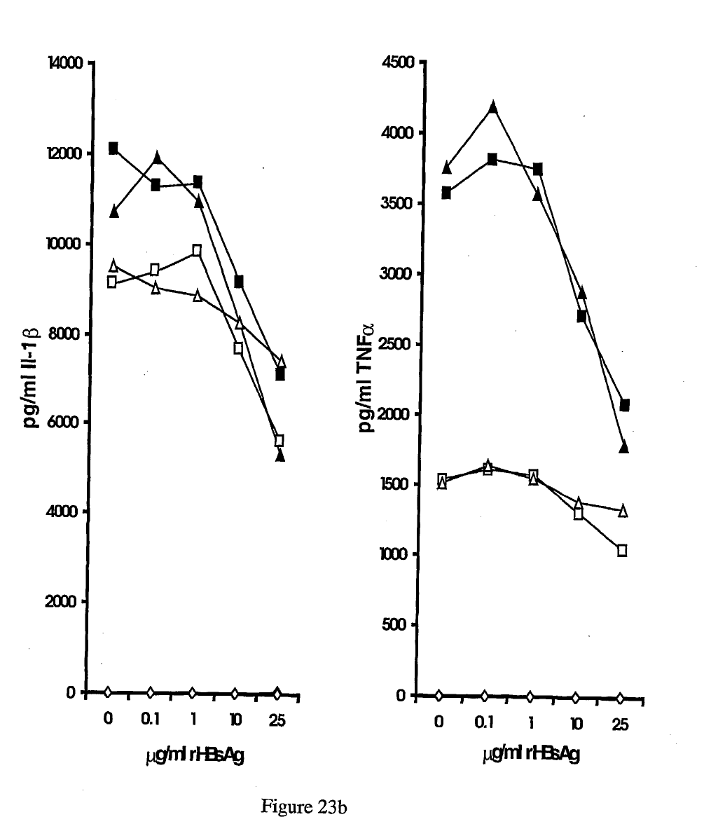

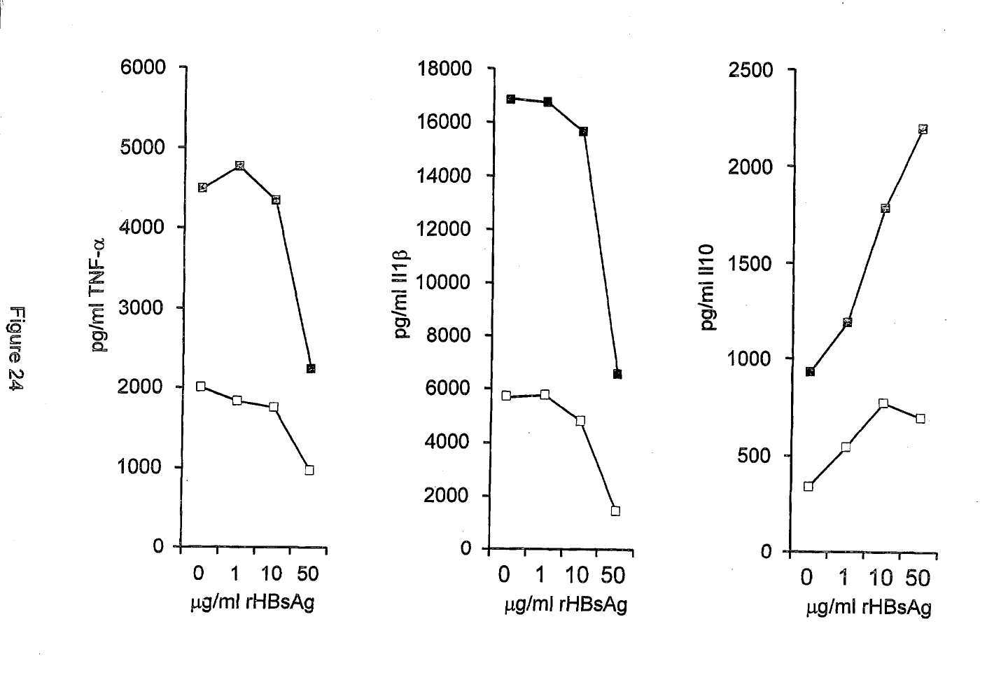

The ability of the anti CD14 antibodies to block the attachment of b-rHBsAg to monocytes was examined. PBMC were incubated with increasing amounts of anti CD14 clone My4 or P9. After washing, the cells were incubated with b-rHBsAg. As shown in FIG. 9a partial inhibition of b-rHBsAg attachment was obtained when cells were pre-incubated with increased concentrations of antibody P9. Even at the lowest concentration of antibody My4 almost complete inhibition of b-rHBsAg binding was obtained (FIG. 9b). A mAb directed against CD18, another LPS binding molecule, had no effect on b-rHBsAg attachment (data not shown). Identical results were obtained when the experiment was performed using biotinylated plasma purified HBsAg (FIG. 9c)

### Example 8

Expression of Human and Mouse CD14 in a Non-Monocytic Cell Line Results in Attachment of b-rHBsAg.

The ability of CD14 to induce binding of b-rHBsAg when expressed in a non-monocytic cell line was examined. CHO cells expressing human CD14 or cells transfected with the empty plasmid were rovided by Dr. Stelter (Jack et al., 1995). As shown in FIG. 10 both CD14 specific mAbs did recognize the CD14 transfected cells and not the control cells. Attachment of b-rHBsAg was also only observed to CHO cells which expressed CD14.

As shown in Table 1, attachment of b-rHBsAg was also observed to CHO cells which expressed mouse CD14. The binding of mAbs specific for human CD14 (P9 and My4) or mouse CD14 (rmC5-3) only to their specific ligand demonstrated that no cross-contamination between the two cell lines had occurred.

### Example 9

Non Biotinylated rHBsAg Can Be Detected on CHO-CD14 Cells by a Serotype ‘d’ Specific Antibody.

To aim of the experiments described here was to demonstrate that indeed rHBsAg particles were binding to the cells and not some unknown (biotinylated) contaminant present in the rHBsAg preparations. First CHO-CD14 cells were incubated with 5 or 25 μg/ml non-biotinylated rHBsAg, followed by different mAbs and anti-mouse-FITC. Cells were analyzed by FACS analysis. As expected rHbsAg was only detected by the mAb specific for the ‘d’ serotype and not by the mAb specific for the ‘y’ serotype (FIG. 11a). No signal was obtained with the isotype control antibody. When CHO-DHFR cells were tested no signal was obtained with the mAb specific for the ‘d’ seroype (FIG. 11b), again demonstrating that attachment of rHBsAg depends on the presence of CD14.

### Example 10

Residues Important for LPS Binding are also Important for Attachment of b-rHBsAg to the Cells.

To identify the region on CD14 involved in the binding of rHBsAg to the cells, inhibition experiments with several Mabs with known epitope specificity were performed (see Table 2). As shown in FIG. 12 mAb My4 blocked binding of b-rHBsAg very efficient. Good inhibition was obtained with 2.5 μg/ml of mAbs biG4 and biG11. Very weak inhibition was observed with mAb P9, while biG2 did not inhibit binding of b-rHBsAg. From this experiment it is concluded that the region of AA 33-44 of CD14 (VSAVEVEIHAGG (SEQ ID NO 2), which is important for binding of LPS, is also involved in the attachment of b-rHBsAg to the cell.

To confirm these results the binding of b-rHBsAg to CD14 mutated at positions 39, 40, 41, 43 and 44 was investigated. This mutant (mut6: Stelter et al., 1997) is efficiently reized by a rabbit anti-CD14 polyclonal serum (Table 3). The mutant is also recognized by several mAbs like P9 and biG2, although binding is reduced compared to the wt CD14. mAb biG4 did not bind to mut6. b-rHBsAg did not bind to CHO cells transfected with mut6, confirming the results obtained with the antibody-inhibition experiments.

### Example 11

The Effect of Serum Concentrations on the Attachment of b-rHBsAq to Monocytes.

All binding experiments described were performed in HBSS containing 1-2% HS, because in initial experiments an enhancement of binding at these concentrations was observed (data not shown). Here the effect of low and high serum concentrations on the attachment of b-rHBsAg to CD14 expressing cells was investigated. As shown in FIG. 13, attachment of b-rHBsAg to monoctes was clearly enhanced at low serum concentrations (1-3%) At higher concentrations (7-10%) binding was even inibited compared to the serum negative control. The ability of serum to induce binding of b-rHBsAg to CD14 when expressed in a non-monocytic cell line was also examined. Attachment of b-rHBsAg to CD14 expressing CHO cells was also enhanced at low serum concentrations (FIG. 14).

Incubation of serum at 56° C. for 30 minutes clearly destroyed the factor which enhanced the attachment of b-rHBsAg to PBMC (FIG. 15), demonstrating the (heat-labile) protein nature of this factor.

Because a soluble form of CD14 is present in circulation the inhibitory effect of the higher serum concentrations might be due to this sCD14. As shown in FIG. 16 a dose dependent inhibition of b-rHBsAg binding was observed when recombinant sCD14 was added. However the amount of sCD14 needed to obtain more then 50% inhibition (>2.5 μg/ml) exceeds the amount of sCD14 in present 10% serum (0.3-0.7 μg/ml). This suggest that other serumfactors exsist that can inhibit the binding of b-rHBsAg to monocytes.

### Example 12

Identification of LPS Binding Protein (LBP) as the Serum Enhancing Factor.

Because human LBP is known to be rapidly inacivated at 56° C. and because of the remarkable similarity between LPS and rHBsAg binding to CD14 positive cells, LBP was considered a likely candidate for being the factor which enhances attachment of rHBsAg to the cells. Indeed, when purified LBP was used instead of serum, binding of b-rHBsAg was strongly enhanced in a dose dependent manner (FIG. 17). Further evidence for the role of LBP was obtained using two different LBP specific mabs. When these were included during incubation of PBMC with b-rHBsAg ih 2% HS, attachment of b-rHBsAg was completely abrogated (FIG. 18). Taken together these experiments show that LBP is indeed the serum factor which enhances binding of rHBsAg to CD14 positive cells.

### Example 13

rHBsAg Can Reduce the Attachment of CD14-Specific mAbs to CHO-CD14

CD14 is a GPI-anchored membrane protein and therefore lacks signalling capacity. So, most probably an additional membrane protein is needed to trigger a suppressive pathway by HBsAg. This suggest that CD14 is not necessarily the final receptor for HBsAg and like LBP rather acts as a shuttle protein. To distinguish between these two possibilities CHO-CD14 cells were pre-incubated with 50 μg/ml rHBsAg in the presence of 0.5 μg/ml LBP and 1% HS. Binding of the different CD14-specific mAbs to these rHBsAg pre-treated cells was compared with the binding to non-rHBsAg treated cells. As shown in FIG. 19 attachment of b-rHbsAg was reduced strongly by pre-incubation with rHBsAg (˜80%), which indicates that most rHBsAg binding sites on the cell were occupied. Only the binding of mAb biG11 was reduced (˜60%) to almost the same level as the reduced binding of b-rHBsAg The attachment of mAbs P9, My4 and biG4 was reduced only slightly (10-30%), while binding of mAb biG2 was enhanced slightly. These results suggest that rHBsAg remains attached to CD14 or is at least in very close proximity of CD14.

### Example 14

Effect of Lipids and Lipid-Reconstituted HBsAn on the Attachment of b-rHBsAG to Monocytes.

It was first investigated if certain lipids could prevent the binding of b-rHBsAg to monocytes. PBMC were incubated with phosphatidylcholine (PC), phosphatidylserine (PS) and phosphatidylglycerol (DOPG). After washings, cells were incubated with b-rHBsAG. As shown in FIG. 20, PS and DOPG inhibited binding of b-rHBsAg very well, while PC did not. Next the inhibitory effect of plasma derived HBsAg, delipidated and PC, PS and DOPG-reconstituted HBsAg was investigated. To saturate all binding sites, 0.5 ug/ml LBP was added to the binding buffer. Compared to rHBsAg the inhibition obtained with HBsAg was not very efficient. We have observed that upon storage of rHBsAg at 4° C., the potential to bind to monocytes gradually dissappears. The plasma purified HBsAg used here has been stored at 4° C. for several months, which might explain the low inhibition observed. However, after delipidation even the small inhibition obtained with HBsAg was no longer observed. Reconstitution of delipidated HBsAg with PS and DOPG resulted in very strong inhibition of b-rHBsAg binding to monocytes. Reconstitution with PC did not have this effect (FIG. 21). Finally the antigenicity of the different preparations was compared (FIG. 22). Recombinant and plasma HBsAg were recognised equally well by all 4 mAbs, despite the major differences in inhibitory activity for b-rHBsAg binding. The antigenicity of delipidated and PC reconstituted HBsAg was very low, while increasing antigenicity was observed for the PS and DOPG reconstituted HBsAg preparations.

### Example 15

Effect of rHBsAq on LPS Induced Cytokine Production.

THP-1 were incubated for 24 h with 100 nM 1,25-VitD3, washed and incubated with or without LPS in the presence of different HBsAg concentrations. Cell supernatant was collected after 24 h of incubation and tested for the presence of IL-1β and TNFα. As shown in FIG. 22 HBsAg alone did not induce any cytokine production while LPS induced the secretion of the 2 cytokines. High levels of TNFα were produced while lower concentrations of IL-1β were detected. Highest cytokine levels were obtained with 50 ng/ml LPS. Cytokine concentrations were dose-dependently decreased in the presence of HBsAg.

The effect of HBsAg on LPS induced cytokine production was also studied with PBMC from two different donors. As shown in FIG. 23 PBMC from donor 1 produced higher amounts of TNFα compared to the PBMC from donor 2. Nevertheless, dose-dependent reduction of TNFα levels in the presence of HBsAg was more pronounced with PBMC form donor 1. Similar results were obtained for IL-1β.In FIG. 24 the results obtained with PBMC from two other donors are shown. Identical results were obtained, while it was also demonstrated that the secretion of the anti-inflammatory cytokine IL-10 was increased.

### Example 16

Effect of rHBsAg on the IL-2 Induced Activation of Monocytes.

The effect of rHBsAg on IL-2 induced cytokine production was studied with PBMC from 4 donors. PBMC were incubated in cRPMI with or without 1000 U/ml IL-2. 0, 1, 10, 25 or 50 μg/ml rHBsAg was added. Cell supernantants were collected after 24 h and tested for the presence of IL-8. As shown in Table 4 non-stimulated PBMC already produced IL-8, the levels of which increased by adding IL-2. In the presence of rHBsAg the IL-8 production of both stimulated and non-stimulated PBMC decreased in a dose-dependent manner. Nevertheless, rHBsAg clearly inhibited the production of IL-8 induced by IL-2.

TABLE 1
b-rHBsAg binds to CHO cells which express mouse CD14.

| | | Median Fluorescence | | |
|---|---|---|---|---|
| | | anti-mCD14 | anti-hCD14 | |
| | b-rHBsAg | rmC5-3 | P9 | My4 |
| hCD14-CHO | 122.66 | 0.00 | 502.44 | 291.20 |
| mCD14-CHO | 48.19 | 23.98 | 0.00 | 0.12 |

Cells were incubated with b-rHBsAg in 2% HS-HBSS or with different FITC-labelled mAbs. After washing, b-rHBsAg was detected with Strep-PE. Median fluorescence was determined.

TABLE 2
Epitope specificity of anti-CD14 mAbs (according to
Stelter et al., 1997).

| mAb | epitope | inhibition of LPS |
|---|---|---|
| biG2 | 147-152 | − |
| biG4 | 33-44 | − |
| biG11 | 39-44 | + |
| My4 | 39-44 | + |
| P9 | 135-152 | − |

Table 3: Substitution of amino acids 39, 40, 41, 43 and 44 of human CD14 with alanine (mut6) abrogates attachment of b-rHBsAg to CHO cells.

Median Fluorescence

| | b-rHBsAg | rabbit anti-CD14 | biG2 | biG4 |
|---|---|---|---|---|
| CHO-CD14 | 122.44 | 261.79 | 601.14 | 1237.79 |
| CHO-Mut6 | 0.92 | 349.98 | 42.58 | 0.00 |

Cells were incubated with b-rHBsAg in 2% HS-HBSS or with different CD14 specific mAbs or a CD14 specific polyclonal serum. After washing, b-rHBsAg was detected with Strep-PE while antibodies were detected with goat anti-rabbit IgG-FITC or rabbit anti-mouse F(ab′)2-FITC. Median fluorescence was determined.

TABLE 4
rHBsAg reduces IL-2 induced IL-8 production.

| | | IL-2 | | |
|---|---|---|---|---|
| PBMC | μg/ml | − | + | IL-2 induced increase |
| donor | rHBsAg | pg/ml IL-8 | | expressed in % |
| a | 0 | 2659 | 4946 | 100 |
| | 1 | 3006 | 4403 | 61 |
| | 10 | 2307 | 4095 | 78 |
| | 25 | 2049 | 3018 | 42 |
| b | 0 | 4993 | 16306 | 100 |
| | 1 | 4393 | 9995 | 88 |
| | 10 | 4750 | 9710 | 44 |
| | 50 | 2347 | 6694 | 38 |
| c | 0 | 13122 | 25889 | 100 |
| | 1 | 6631 | 18188 | 91 |
| | 10 | 5280 | 13187 | 62 |
| | 50 | 4019 | 14576 | 83 |
| d | 0 | 906 | 1428 | 100 |
| | 1 | 941 | 1419 | 92 |
| | 10 | 997 | 1296 | 57 |
| | 25 | 999 | 1168 | 32 |

The IL-2 induced increase of IL-8 levels was determined by first substracting IL-8 concentrations of non-stimulated cells from stimulated ones. These values were compared to the one of the non-rHBsAg treated PBMC which was set at 100%.

References

Adkins, J. and Wagstaff, A. (1998). Recombinant Hepatitis B vaccine. A review of its immunogenecity and protective efficacy against hepatitis B. Biodrugs 10, 137-158

Bazil, V., and Strominger, J. L. (1991). Shedding as a mechanism of down-modulation of CD14 on stimulated human monocytes. J Immunol 147(5), 1567-74.

Beutler, B., Milsark I., and Cerami, A. (1985). Passive immunization against cachectin/tumor necrosis factor protects mice from lethal effect of endotoxin. Science 229(4716), 869-71

Blum, H., Stowring, L., Figus, A., Montgomery, C., Haase, A., and Vyas, G. (1983). Detection of HBV DNA in hepatocytes, bile duct epithelium and vascular elements in in situ hybridization. Proc Natl Acad Sci 80, 6685-88.

Blumberg, B. (1997). Hepatitis B virus, the vaccine, and the control pf primary cancer of the liver. Proc. Natl. Acad. Sci. USA 94, 7121-5.

Bosco, M. C., Espinoza-Delgado, I., Rowe, T. K., Malabarba, M. G., Longo, D. L., and Varesio, L. (1997). Functional role for the myeloid differentiation antigen CD14 in the activation of human monocytes by IL-2. J immunol 159(6), 2922-31.

Budkowska, A., Bedossa, P., Groh, F., Louise, A., and Pillot, J. (1995). Fibronectin of human liver sinusoids binds hepatitis B virus: identification by an anti-idiotypic antibody bearing the internal image of the pre-S 2 domain. J Virol 69, 840-48.

Bufler, P., Stiegler, G., Schuchmann, M., Hess, S., Krüger, S., Stelter, F., Eckerskorn, C., Schütt, C, and Engelmann, H. (1995). Soluble lipopolysaccharide receptor (CD14) is released via two different mechanisms from human monocytes and CD14 transfectants. Eur. J. Immunol.25, 604-610.

Cavanaugh, V., Guidotti, L., and Chisari (1998). Inhibition of hepatitis B virus replication during adenovirus and cytomegalovirus infections in transgenic mice. J Virol 72, 2630-7.

Dejean, A., Lugassy, C., Zafrani, S., Tiollais, P., and Brechot, C. (1984). Detection of hepatitis B virus DNA in pancreas, kidney and skin of two human carriers of the virus. J Gen Virol 65, 651-55.

Devitt, A., Moffatt, O. D., Raykundalia, C., Capra, J. D., Simmons, D. L., and Gregory, C. D. (1998). Human CD14 mediates recognition and phagocytosis of apoptotic cells. Nature 392, 505-509.

Ellis, R. W. (1993) “Hepatitis B vaccines in Clinical practice” edited by Marcel Dekker, Inc, New York, USA.

Fadok, V. A. (1998). Macrophages that have ingested apoptotic cells in vitro inhibit proinflammatory cytokine production through autocrine/paracrine mechanisms involving TGF-β, PGE 2 and PAF. J Clin Invest 101, 890-98.

Fearns, C., Kravchenko, V., Ulevitch, R., and Loskutoff, D. (1995). Murine CD14 gene expression in vivo: extramyeloid synthesis and regulation by lipoploysaccharide. J. Exp. Med. 181, 857.

Ferrari, C., Penna, A., Bertoletti, A., Valli, A., Antoni, A. D., Giuberti, T., Cavalli, A., Petit, M. A., and Fiaccadori, F. (1990). Cellular immune response to hepatitis B virus-encoded antigens in acute and chronic hepatitis B virus infection. J Immunol 145(10), 3442-49.

Ferrero, E., Hsieh, C., Francke, U., and Goyert, S. (1990). CD14 ids a member of the family of leucine-rich proteins and is encoded by a gene syntenic with multiple receptor genes. J Immunol 145, 331-36.

Ferrero, E., Jiao, D., Tsuberi, B., Tesio, L., Rong, G., Haziot, A., Goyert, S. (1993) Transgenic mice expressing human CD14 are hypersensitive to lipopolysaccharide. Proc. Natl. Acad. Sci. USA 90, 2380-4

Franco, A., Paroli, M., Tresta, U., Benvenuto, R., Peschle, C., Balsano, F., and Bamaba, V. (1992). Transferrin receptor mediates uptake and presentation of hepatitis B envelope antigen by T lymphocytes. J Exp Med 175, 1095-105.

Gerlich, W. and Bruss, V. F (1993). Functions of hepatitis B virus proteins and molecular targets for protective immunity. In “Hepatitis B vaccines in Clinical practice” edited by Ellis, R. W. Marcel Dekker, Inc, New York, USA.

Gerlich, W. H., Lu, X., and Heermann, K. H. (1993b). Studies on the attachment and penetration of hepatitis B virus. J Hepatol 17(3), S10-14.

Golenbock, D.,, Liu, Y., Miliham, F., Freeman, M., and Zoeller, R. (1993). Surface expression of human CD14 in Chinese hamster ovary fibroblasts imparts macrophage-like responsiveness to bacterial endotoxin. J. Biol. Chem. 268, 22055-9

Goyert, S., Ferrero, E., Seremitis, S., Winchester, R., Silver, J. and Mattison, A., (1986). Biochemistry and expression of myelomonocytic antigens. J. Immunol, 137, 3909.

Hagelstein, J., Fathinejad, F., Stremmel, W., and Galle, P. R. (1997). pH-independent uptake of hepatitis B virus in primary human hepatocytes. Virology 229, 292-94.

Harrison, T. J. (1990). Hepatitis B virus DNA in peripheral blood leukocytes: a brief review. Journal of Medical Virology 31(1), 33-35.

Haziot, A., Chen, E., Ferrero, E., Low, M. G., Silber, R., and Goyert, S. M. (1988). The monocyte differentiation antigen, CD14, is anchored to the cell membrane by a phosphatidylinositol likage. J Immunol 141, 547-52.

Heidenreich, S., Schmidt, M., August, C., Cullen, P., Rademaekers, A., and Pauels, H. G. (1997). Regulation of human monocyte apoptosis by the CD14 molecule. J Immunol 159(7), 178-88.

Hertogs, K., Leenders, W. P. J., Depla, E., De Bruin, W. C. C., Meheus, L., Raymackers, J., Moshage, H., and Yap, S. H. (1993). Endonexin II, present on human liver plasma membranes, is a specific binding protein of small hepatitis B virus (HBV) envelope protein. Virology 197, 549-57.

Hetherington, C. J., Kingsley, P. D., Crocicchio, F., Zhang, P., Rabin, M. S., Palis, J., and Zhang, D. E. (1999). Characterization of human endotoxin lipopolysaccharide receptor CD14 expression in transgenic mice. J Immunol 162, 503-9.

Heumann, D., Barras, C., Severin, A., Glauser, M. P., and Tomasz, A. (1994). Gram positive cell walls stimulate synthesis of tumor necrosis factor alpha and interleukin-6 by human monocytes. Infect Immun 62, 2715-21.

Imai, M., Yanase, Y., Nojiri, T., Miyakawa, Y., and Mayumi, M. (1979). A receptor for polymerized human and chimpanzee albumins on hepatitis B virus particles co-occurring with HBeAg. Gastroenterology 76, 242-47.

Jabara, H. H., and Vercelli, D. (1994). Engagement of CD14 on monocytes inhibits the synthesis of human Igs, including IgE. J Immunol 153(3), 972-78.

Jack, R. S., Grunwald, U., Stelter, F., Workalemahu, G., and Schutt, C. (1995). Both membrane-bound and soluble forms of CD14 bind to gram-negative bacteria. European Journal of Immunology 25(5), 1436-1441.

Komai, K., and Peeples, M. E. (1990). Physiology and function of the vero cell receptor for the hepatitis B virus small S protein. Virology 177, 332-38.

Korba, B. E., Cote, P. J., Wells, F. V., Baldwin, B., Popper, H., Purcell, R. H., Tennant, B. C., and Gerin, J. L. (1989). Natural history of woodchuck hepatitis virus infections during the course of experimental viral infection: Molecular virologic features of the liver and lymphoid tissues. J Virol 63, 1360-70.

Kravchenko, V., Steinemann, S., Kline, L., Feng, L., and Ulevitch, R. (1996) Endotoxin tolerance is induced in Chinese hamster ovary cell lines expressing human CD14.Shock 5, 194-201.

Kusunoki, T., Hailman, E., Juan, T. S., Lichenstein, H. S., and Wright, S. D. (1995). Molecules from Staphylococcus aureus that bind CD14 and stimulate innate immune responses. J Exp Med 182, 1673-82.

Labeta, M. O., Landmann, R., Obrecht, J. P., and Obrist, R. (1991). Human B cells express membrane-bound and soluble forms of the CD14 myeloid antigen. Mol Immunol 28(1-2), 115-22.

Lu, X., Block, T. M., and Gerlich, W. H. (1996). Protease-induced infectivity of hepatitis B virus for a human hepatoblastoma cell line. J Virol 70(4), 2277-85.

Lue, K. H., Lauener, R. P., Winchester, R. J., Geha, R. S., and Vercelli, D. (1991). Engagement of CD14 on human monocytes terminates T cell proliferation by delivering a negative signal to T cells. J Immunol 147, 1134.

Malmqvist, M. (1999). BIACORE: an affinity biosensor system for characterization of biomolecular interactions.

Mason, A., Wick, M., White, H., and Perrillo, R. (1993). Hepatitis B virus replication in diverse cell types during chronic hepatitis B virus infection. Hepatology 18(4), 781-89.

Matsuura, K., Setoguchi, M., Nasu, N., Higuchi, Y., Yoshida, S., Akizuki, S., and Yamamoto, S. (1989). Nucleotide and amino acid sequences of the mouse CD14 gene. Nucleic Acids Res. 17, 2132.

Mehdi, M., Kaplan, M. J., Anlar, F. Y., Yang, X., Bayer, R., Sutherland, K., and Peeples, M. E. (1994). Hepatitis B virus surface antigen binds to apolipoprotein H. J Virol 68(4), 2415-24.

Mehdi, H., Yang, X., and Peeples, M. E. (1996). An altered form of apolipoprotein H binds hepatitis B virus surface antigen most efficiently. Virology 217, 58-66.

Moshage, H. (1997). Cytokines and the hepatic acute phase response. J Pathol 181, 257.

Muller, C., and Zielinski, C. C. (1990). Impaired lipopolysaccharide-inducible tumor necrosis factor production in vitro by peripheral blood monocytes of patients with viral hepatitis. Hepatology 12(5), 1118-24.

Muller, C., and Zielinski, C. C. (1992). lnterleukin-6 production by peripheral blood monocytes in patients with chronic liver disease and acute viral hepatitis. J Hepatol 15(3), 372-77.

Nagaraju, K., Naik, S. R., and Naik, S. (1998). Chronic hepatitis B virus carriers have low lymphoproliferative responses to HBsAg and reduced interleukin-2 synthesis. Indian J Gastroenterol 17(3), 83-86.

Neurath, A. R., Strick, N., Sproul, P., Ralph, H. E., and Valinsky, J. (1990). Detection of receptors for hepatitis B virus on cells of extrahepatic origin. Virology 176, 448-57.

Neurath, A. R., Strick, N., and Sproul, P. (1992). Search for hepatitis B virus cell receptors reveals binding sites for interleukin 6 on the virus envelope protein. J Exp Med 175, 461-69.

Newman, S. L., Chaturvedi, S., and Klein, B. (1995). The W-1 antigen of blastomyces dermatitidis yeasts mediates binding to human macrophage CD11b/DC18 (CR3) and CD14. J Immunol 154, 753-61.

Papaevangelou, G. (1998). Current combined vaccines with hepatits B. Vaccine 16suppl, 69-72.

Paulij, W. P., de Wit, P. L. M., Sunnen, C. M. G., van Roosmalen, M. H., Petersen-van Ettekoven, A., Cooreman, M. P. and Heijtink, R. A. (1999). Localization of a unique hepatitis B virus epitope sheds new light on the structure of hepatitis B virus surface antigen. J. Gen. Virol. 80: 2121-2126.

Pedron, T., Girard, R., and Chaby, R. (1995). Variation of LPS-binding capacity, epitope expression, and shedding of membrane-bound CD14 during differentiation of human monocytes. J Immunol 155, 1460-71.

Petit, M. A., Capel, F., Dubanchet, S., and Mabit, H. (1992). PreS1-specific binding proteins as potential receptors for hepatitis B virus in human hepatocytes. Virology 187, 211-22.

Pontisso, P., Morsica, G., Ruvoletto, M. G., Zambello, R., Colletta, C., Chemello, L., and Alberti, A. (1991). Hepatitis B virus binds to peripheral blood mononuclear cells via the pre S1 protein. J Hep 12, 203-6.

Pontisso, P., Ruoletto, M., Tiribelli, C., Gerlich, W., Ruol, A., and Alberti, A. (1992). The preS1 domain of hepatitis B virus and IgA cross-react in their binding to the hepatocyte surface. J Gen Virol 73, 2041-45.

Scott,M. G, Vreugdenhil, A. C. E., Buurman,W. A., Hancock,R. E. W., and Gold, M. R. (2000). Cutting edge: Cationic antimicrobial peptides block the binding of lipolysaccharide (LPS) to LPS binding protein. J. Imunol. 164, 549-553.

Setoguchi, M., Nasu, N., Yoshida, S., Higuchi, Y. Akizuki, S., and Yamamoto, S. (1989). Mouse and human CD14 (myeolid cell-specific leucine-rich glycoprotein) primary structure deduced from cDNA clones. Biochim. Biophys. Acta 1008, 213-22.

Soell, M., Lett, E., Hoveck, F., Scholler, M., Wachsmann, D., and Klein, J. (1995). Activation of human monocytes by streptococcal rhamnose glucose polymers is mediated by CD14 antigen and mannan binding protein inhibits TNF-alpha release. J Immunol 154, 852-60.

Stelter, F., Pfister, M., Bernheiden, M., Jack, R., Butler, P., Engelmann, H., and Schutt, C. (1996). The myeloid differentiation antigen CD14 is N- and O-glycosylated. Contribution of N-linked glycosylation to different soluble CD14 isoforms. Eur. J. Biochem. 236, 457-64.

Stelter, F, Bernheiden, M., Menzel, R., Jack, R., Witt, S., Fan, W., Pfister, M. and Schütt, C. (1997). Mutation of amino acids 39-44 of human CD14 abrogates binding of lipopolysaccharide and Eschericia coli. Eur. J. Biochem. 243, 100-109.

Stirok, H. J, Thornton, J. M. and Howard, C. R. (1992). A topological model for hepatitis B surface antigen. Intervirology 33: 148-158

Stricker, E. A. M., Tiebout, R. F., Lelie, P. N., Zeijlemaker, W. P. (1985). A human monoclonal IgG1λ anti-hepatitis B surface antibody. Production, properties and application. Scand. J. Immunol. 22, 337-343.

Su, G. L., Dorko, K., Strom, S. C., Nussler, A. K., and Wang, S. C. (1999). CD14 expression and production by human hepatocytes. J Hepatol 31, 435-42.

Tamura, Y., Higuchi, Y., Kataoka, M., Akizuki, S., Matsuura, K., and Yamamoto, S. (1999). CD14 transgenic mice expressing membrane and soluble forms: comparisons of levels of cytokines and lethalities in response to lipopolysaccharide between transgenic and non-transgenic mice. Int. Immunol. 11,333-9

Treichel, U., Meyer zum Buschenfelde, K. H., Stockert, R. J., Poralla, T., and Gerken, G. (1994). The asialoglycoprotein receptor mediates hepatic binding and uptake of natural hepatitis B virus particles derived from viraemic carriers. J Gen Virol 75, 3021-29.

Trippler, M., Meyer zum Buschenfelde, K. H., and Gerken, G. (1999). HBV viral load within subpopulations of peripheral blood mononuclear cells in HBV infection using limiting dilution PCR. J Virol Meth 78, 129-47.

Ulevitch R., Mathison J., Schumann R., and Tobias P. (1990). . A new model of macrophage stimulation by bacterial lipopolysaccharide. J Trauma 30(12 Suppl), S189-92.

Ulevitch, R. and Tobias, P. (1999). Recognition of Gram-negative bacteria and endotoxin by the innate immune system. Curr. Opinion in Immunol. 11, 19-22.

Vanlandschoot, P., Beirnaert, E., Barrere, B., Calder, L., Millar, B., Wharton, S., Min Jou, W., and Fiers, W. (1998). An antibody which binds to the membrane proximal end of influenza haemagglutinin (H3 subtype) inhibits the low-pH-induced conformational change and cell-cell fusion but does not neutralize virus. J. Gen. Virol. 79, 1781-1791.

Vingerhoets, J., Michielsen, P., Vanham, G., Bosmans, E., Paulij, W., Ramon, A., Pelckmans, P., Kestens, L., and Leroux-Roels, G. (1998). HBV-specific lymphoproliferative and cytokine responses in patients with chronic hepatitis B. J Hepatol 28(1), 8-16.

Voll, R. E. (1997). Immunosuppressive effects of apoptotic cells. Nature 390, 350-351.

Wright, S. D., Ramos, R. A., Tobias, P. S., Ulevitch, R. J., and Mathison, J. C. (1990). CD14, a receptor for complexes of lipopolysaccharide (LPS) and LPS binding protein. Science 249, 1431.

Yoffe, B., Burns, D. K., Bhatt, H. S., and Combes, B. (1990). Extrahepafic hepatitis B virus DNA sequences in patients with acute hepatitis B infection. Hepatology 12, 187-92.

Zeldis, J. B., Mugishima, H., Steinberg, H. N., Nir, E., and Gale, R. P. (1986). In vitro hepatitis B virus infection of human bone marrow cells. Journal of Clinical Investigation 78(2), 411-417.

1. Method for determining the binding between CD14 and Hepatitis B virus components comprising the use of:
a) CD14, or a mutein, or a fragment of CD14 which is able to bind to HBV antigens or particles,
b) HBV components,
c) a ligand or antisense peptide of the molecules mentioned in a) or b),
d) an antibody raised against any of the molecules mentioned in a) or b),
e) an antibody raised against the ligand mentioned in c),
f) an anti-idiotype antibody raised against the antibody mentioned in d), or
g) an anti-idiotype antibody raised against the antibody mentioned in e).

2. Composition for preventing, treating or alleviating HBV infections comprising as an active substance at least one of the following:
a) CD14 or a mutein or fragment thereof which binds to HBV components,
b) a ligand or antisense peptide of the molecules mentioned in a),
c) an antibody raised against the molecules mentioned in a),
d) an antibody raised against the ligand or antisense peptide mentioned in b),
e) an anti-idiotype antibody raised against the antibody mentioned in c), or
f) an anti-idiotype antibody raised against the antibody mentioned in d),

3. A composition according to claim 2 wherein the CD14 fragment which binds to HBV components is a peptide of 6 to 50 amino acids comprising the sequence as represented in SEQ ID NO 1.

4. A composition for preventing, treating or alleviating HBV infections comprising as an active substance an antibody against a ligand of HBV components, wherein said ligand is the LPS binding protein.

5. A composition for preventing, treating or alleviating HBV infections comprising as an active substance a peptide which blocks the binding of the LPS binding protein to HBV components.

6. Vaccine composition for preventing, treating or alleviating HBV infections comprising as an active substance at least one of the following:
a) an antibody raised against a ligand of HBV components as defined in claim 1, wherein said ligand is the LPS binding protein, or,
b) an antibody raised against CD14.

7. Use of HBV components for preventing, treating or alleviating inflammatory reactions.

8. Use of HBV components for the preparation of a medicament for preventing, treating or alleviating inflammatory reactions.

9. Use according to claim 8 wherein said HBV components are reconstituted PS, DOPG or PI HBsAg preparations.

10. A host cell transformed with a nucleic acid sequence coding for a fragment of CD14 which binds to HBV components, said fragment comprising the sequence as represented in SEQ ID NO 1, and with said host expressing said fragment on its surface.

11. Method for identifying compounds interfering with the interaction between CD14 and HBV components comprising the steps of:
a) contacting the compound to be screened with a complementary set of molecules as defined in claim 1, and,
b) detecting the binding affinity of said complementary set of molecules as defined in claim 1 in the presence or absence of said compound to be tested.

12. Method for identifying compounds interfering with the interaction between CD14 and HBV components comprising the steps of:
a) contacting the compound to be screened with a host of claim 10,
b) detecting the formation of a complex between said compound and said CD14 molecule or mutein or fragment thereof,
c) providing HBV components, and
d) detecting the interference of said compound with the binding of HBV components with said CD14 molecule or a mutein or a fragment thereof.

13. Method for identifying compounds interfering with the interaction between HBV-components and LPS binding protein comprising the steps of:
a) contacting a compound to be screened with a host of claim 10, HBV components and LPS binding protein,
b) detecting the interference of said compound with the binding of HBV components with said CD14 molecule or a mutein or a fragment thereof, and,
c) detecting a complex formation between said compound and HBV components or LPS binding protein.

14. Compound obtained by any of the methods of claims 11 to 13 or 24.

15. Compound according to claim 14, further modified for use in a delivery system.

16. Use of a compound according to claim 14 or 15 for preventing, treating or alleviating inflammatory reactions.

17. Use of a compound according to claim 14 or 15 for the preparation of a medicament for preventing, treating or alleviating inflammatory reactions in patients.

18. Use of a compound according to claim 14 or 15 or a molecule as defined in claim 1 or 2 for preventing, treating or alleviating HBV infections.

19. Use of a compound according to claim 14 or 15 or a molecule as defined in claim 1 or 2 for the preparation of a medicament for preventing, treating or alleviating HBV infections.

20. Method for the production of a composition comprising mixing the compound according to claim 14 or 15 with a pharmaceutically acceptable carrier.

21. Use of HBV components in combination with LPS binding protein for preventing, treating or alleviating inflammatory reactions.

22. Use of HBV components in combination with LPS binding protein for the preparation of a medicament for preventing, treating or alleviating inflammatory reactions.

23. Use according to claim 22 wherein said HBV components are reconstituted PS, DOPG or PI HBsAg preparations.

24. Method for identifying compounds interfering with the interaction between CD14 and HBV components comprising the steps of:
a) contacting the compound to be screened with a host of claim 10,
b) detecting the formation of a complex between said compound and said CD14 molecule or mutein or fragment thereof,
c) providing HBV components in combination with LBP, and,
d) detecting the interference of said compound with the binding of HBV components with said CD14 molecule or a mutein or a fragment thereof.
